(19) United States
(12) Patent Application Publication
(10) Pub. No.: US 2022/0218997 A1
(43) Pub. Date: Jul. 14, 2022

(54) SYSTEM AND METHOD FOR CARDIAC CONTROL

(71) Applicant: Bios Health Ltd, Cambridge, Cambridgeshire (GB)

(72) Inventors: Oliver ARMITAGE, Cambridge (GB); Emil HEWAGE, Cambridge (GB); Tristan EDWARDS, Cambridge (GB); Pascal FORTIER-POISSON, Cambridge (GB); Sudhakaran SHUNMUGAM, Cambridge (GB); Matjaz JAKOPEC, Cambridge (GB); Samuel GONSHAW, Cambridge (GB)

(21) Appl. No.: 17/615,036

(22) PCT Filed: May 29, 2020
(86) PCT No.: PCT/GB2020/051307

§ 371 Date: Nov. 29, 2021

(30) Foreign Application Priority Data
May 29, 2019 (GB) 1907605.8

(51) Int. Cl. A61N 1/36

(52) U.S. Cl. CPC A61N 1/36139; A61N 1/36053; A61N 1/36117; A61N 1/36062; A61N 1/36057

(57) ABSTRACT

A system for controlling a cardiac system of a subject comprising: a plurality of sensors arranged to detect physiological activity in a subject and produce physiological signals corresponding to the detected physiological activity; at least one controller arranged to receive the physiological signals and to process the physiological signals using at least one model to determine at least one output signal; and a plurality of neural stimulators arranged to receive the at least one output signal and to provide neural stimulation to the nervous system of the subject based on the at least one output signal.

The present application relates to a system and method for cardiac control, and in particular to a system and method for closed loop cardiac control.

### BACKGROUND

A number of electronic devices have been used and proposed to control or regulate cardiac function in the human or animal body.

There are a range of pacemakers comprising active electronic devices which are implanted in the human or animal body and are arranged to electrically stimulate the heart muscle directly using electrodes which terminate at the heart muscle, for example at the junction of the cardiac nerves with the heart muscle, in order to provide regulation of heart beat to a predetermined rate.

There are also a range of implanted active electronic devices which are implanted in the human or animal body and are arranged to control cardiac function by electrically stimulating the Vagus nerve using implanted electrodes. By applying appropriate forms of stimulation the stimulation may be arranged to drive cardiac function, as may be desired in subjects suffering from heart failure, or to reduce cardiac function, as may be desired in subjects suffering from hypertension.

Currently these devices which provide stimulation without any responsiveness to activity such as bodily variables that provide information about a subjects condition, or neural activity encoding information about the subjects natural control of cardiac function. For example treatment for hypertension (high blood pressure) may be provided by devices which provide stimulation at predetermined times without any responsiveness to a subject's measured blood pressure or neural activity encoding information about the subject brain's natural control of blood pressure.

It is desirable to be able to control cardiac function of a human or animal body in a manner more responsive to bodily activity in real time, or close to real time, in order to provide improved effectiveness.

The embodiments described below are not limited to implementations which solve any or all of the disadvantages of the known approaches described above.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter; variants and alternative features which facilitate the working of the invention and/or serve to achieve a substantially similar technical effect should be considered as falling into the scope of the invention disclosed herein.

In a first aspect, the present disclosure provides a system for controlling a cardiac system of a subject, comprising: a plurality of sensors arranged to detect physiological activity in a subject and produce physiological signals corresponding to the detected physiological activity; at least one controller arranged to receive the physiological signals and to process the physiological signals using at least one model to determine at least one output signal; and a plurality of neural stimulators arranged to receive the at least one output signal and to provide neural stimulation to the nervous system of the subject based on the at least one output signal.

In a second aspect, the present disclosure provides a system comprising a closed loop cardiac function control system according to the first aspect and an external system.

In a third aspect, the present disclosure provides a method for carrying out closed loop cardiac function control comprising; implanting a system according to the first aspect into a body of a subject; and operating the system.

In a fourth aspect, the present disclosure provides a system configured to modulate efferent neural activity of at least one cardiac sympathetic nerve of a subject, the system comprising: at least one controller arranged to determine at least one output signal; and a plurality of neural stimulators arranged to apply the at least one output signal to the at least one cardiac sympathetic nerve of the subject; wherein the at least one output signal modulates the efferent neural activity of the at least one cardiac sympathetic nerve to produce a physiological response in the subject.

In a fifth aspect, the present disclosure provides a system configured to modulate efferent neural activity of at least one renal sympathetic nerve of a subject, the system comprising: at least one controller arranged to determine at least one output signal; and a plurality of neural stimulators arranged to apply the output signal to the at least one renal sympathetic nerve of the subject; wherein the output signal modulates the efferent neural activity of the at least one renal sympathetic nerve to produce a physiological response in the subject.

In a sixth aspect, the present disclosure provides a system configured to modulate efferent neural activity of at least one cardiac parasympathetic nerve of a subject, the system comprising: at least one controller arranged to determine at least one output signal; and a plurality of neural stimulators arranged to apply the output signal to the at least one cardiac parasympathetic nerve of the subject; wherein the output signal modulates the efferent neural activity of the at least one cardiac parasympathetic nerve to produce a physiological response in the subject.

In a seventh aspect, the present disclosure provides a system configured to modulate afferent neural activity of at least one nerve associated with baroreceptors of a subject, the system comprising: at least one controller arranged to determine at least one output signal; and a plurality of neural stimulators arranged to apply the output signal to the at least one nerve associated with at least one baroreceptor of the subject; wherein the output signal modulates the afferent neural activity of the at least one nerve associated with the at least one baroreceptor to produce a physiological response in the subject.

In an eighth aspect, the present disclosure provides a system configured to determine cardiac activity of a subject, the system comprising: at least one neural transducer arranged to receive efferent neural activity of at least one cardiac sympathetic nerve of the subject, and to produce neural data signals derived from the received efferent neural activity; at least one processor arranged to process the neural data signals to provide processed neural data signals, and to use the processed neural data signals to determine cardiac function of the subject.

In a ninth aspect, the present disclosure provides a system configured to determine cardiovascular activity of a subject, the system comprising: at least one neural transducer arranged to receive efferent neural activity of at least one renal sympathetic nerve of the subject, and to produce neural data signals derived from the received efferent neural activity; at least one processor arranged to process the neural data signals to provide processed neural data signals, and to use the processed neural data signals to determine cardiovascular activity of the subject.

In a tenth aspect, the present disclosure provides a system configured to determine cardiovascular activity of a subject, the system comprising: at least one neural transducer arranged to receive afferent neural activity of at least one renal nerve of the subject, and to produce neural data signals derived from the received efferent neural activity; at least one processor arranged to process the neural data signals to provide processed neural data signals, and to use the processed neural data signals to determine cardiovascular activity of the subject.

In an eleventh aspect, the present disclosure provides a system configured to receive signals associated with cardiac function of a subject, the system comprising: at least one sensor arranged to produce at least one signal associated with blood pressure rise and fall of the subject; and at least one sensor arranged to produce at least one signal associated with efferent neural activity to the heart of the subject; wherein the system is arranged to register the timing and magnitude of changes in blood pressure; wherein the system is arranged to register the timing and magnitude of natural efferent neural signals to the heart; wherein the system is arranged to determine a relationship between timing of the natural efferent neural signals to the heart and timing of any corresponding blood pressure change; and wherein the system is arranged to determine a relationship between a magnitude of the natural efferent neural signals and a magnitude of any corresponding blood pressure change.

In a twelfth aspect, the present disclosure provides a method of determining baroreceptor sensitivity of a subject, the method comprising: receiving at least one signal associated with blood pressure rise and fall of the subject; and receiving at least one signal associated with efferent neural activity to the heart of the subject; registering the timing and magnitude of changes in blood pressure; registering the timing and magnitude of natural efferent neural signals to the heart; determining a timing relationship between timing of the natural efferent neural signals to the heart and timing of any corresponding blood pressure change; and determining a magnitude relationship between a magnitude of the natural efferent neural signals and a magnitude of any corresponding blood pressure change; and determining a baroreceptor sensitivity using the determined timing relationship and magnitude relationship.

The methods described herein may be performed by software in machine readable form on a tangible storage medium e.g. in the form of a computer program comprising computer program code means adapted to perform all the steps of any of the methods described herein when the program is run on a computer and where the computer program may be embodied on a computer readable medium. Examples of tangible (or non-transitory) storage media include disks, thumb drives, memory cards etc. and do not include propagated signals. The software can be suitable for execution on a parallel processor or a serial processor such that the method steps may be carried out in any suitable order, or simultaneously.

This application acknowledges that firmware and software can be valuable, separately tradable commodities. It is intended to encompass software, which runs on or controls “dumb” or standard hardware, to carry out the desired functions. It is also intended to encompass software which “describes” or defines the configuration of hardware, such as HDL (hardware description language) software, as is used for designing silicon chips, or for configuring universal programmable chips, to carry out desired functions.

The features of each of the above aspects and/or embodiments may be combined as appropriate, as would be apparent to the skilled person, and may be combined with any of the aspects of the invention. Indeed, the order of the embodiments and the ordering and location of the preferable features is indicative only and has no bearing on the features themselves. It is intended for each of the preferable and/or optional features to be interchangeable and/or combinable with not only all of the aspect and embodiments, but also each of preferable features.

### BRIEF DESCRIPTION OF THE DRAWINGS

Embodiments of the invention will be described, by way of example, with reference to the following drawings, in which:

FIG. 1 is a schematic diagram of a first example of a cardiac control system according to a first embodiment;

FIG. 2 is a more detailed diagram of a controller part of the system of the cardiac control system of FIG. 1;

FIG. 3 is a schematic diagram of a second example of a cardiac control system according to a first embodiment;

FIG. 4 is a schematic diagram of a third example of a cardiac control system according to a first embodiment;

FIG. 5 is a schematic diagram of a fourth example of a cardiac control system according to a first embodiment;

FIG. 6 is a schematic diagram of a fifth example of a cardiac control system according to a first embodiment;

FIG. 7 is an explanatory diagram representing an example of data flows in the neural control system of the first embodiment;

FIG. 8 is a schematic diagram of an example of a cardiac control system according to a second embodiment; and

FIG. 9 is an explanatory diagram of a method of determining baroreceptor sensitivity.

Common reference numerals are used throughout the figures to indicate similar features. It should however be noted that even where reference numerals for features used throughout the figures vary, this should not be construed as non-interchangeable or distinct. Indeed, unless specified to the contrary, all features referring to similar components and/or having similar functionalities of all embodiments are interchangeable and/or combinable.

### DETAILED DESCRIPTION

Embodiments of the present invention are described below by way of example only. These examples represent the best ways of putting the invention into practice that are currently known to the Applicant although they are not the only ways in which this could be achieved. The description sets forth the functions of the example and the sequence of steps for constructing and operating the example. However, the same or equivalent functions and sequences may be accomplished by different examples.

It should be noted that although exemplary examples, descriptions and/or embodiments are provided under separate headings, these headings should simply serve as a reading aid to provide structure to the description. For the avoidance of any doubt, the features described in any embodiment and/or the embodiments themselves are combinable with the features of any other embodiment and/or any other embodiment unless express statement to the contrary is provided herein. Simply put, the features described herein are not intended to be distinct or exclusive but rather complementary and/or interchangeable.

The present disclosure provides a cardiac control system using machine learning techniques to analyze neural data to determine, in real time, at least one output neural stimulus required to bring the bodily variable into agreement with, or at least closer to, a desired bodily state, and generating the output neural stimulus, and so provide a closed loop neural control system.

The present disclosure also provides cardiac control systems and methods providing improved real time performance.

It should be understood that the nervous system of mammals, such as humans, is generally made up of nerves comprising a plurality of neurons and consists of two main parts: the central nervous system (CNS) and the peripheral nervous system (PNS). In most animals and humans, herein referred to as a subject, the CNS includes the brain and the spinal cord, which are made up of special nerves. The PNS includes the somatic nervous system (SoNS) and the autonomic nervous system (ANS), which are made up of many different types of nerves such as, by way of example only but not limited to, afferent nerves (e.g. sensory nerves), efferent nerves (e.g. control nerves), and/or mixed nerves. The SoNS may carry, by way of example only but is not limited to, conscious motor control for motion and sensation. The ANS may carry, by way of example only but is not limited to, unconscious organ control or unconscious control of bodily functions of the subject.

The SoNS is associated with voluntary control of body movements (e.g. control of skeletal muscles). For example, in the SoNS, afferent nerves include sensory neurons and are responsible for relaying sensation from the body to the CNS and efferent nerves include non-sensory neurons and are responsible for sending out neural information, commands, intent, which may also be referred to as bodily variables as described below, from the CNS to the body (e.g. stimulating muscle contraction). The ANS includes, by way of example only but is not limited to, the sympathetic nervous system (SNS), the parasympathetic nervous system (PSNS) and the enteric nervous system (ENS).

The ANS being associated with organ control of the subject and maintaining homeostasis, the components of the ANS, the SNS and PSNS, are associated with the fight and flight & CHILL ME OUT mechanisms of organs respectively. The SNS largely acts to upregulate organ function where as the PSNS largely acts to downregulate organ function. Nerve fibres in the SNS are primarily routed from the brain stem down through the spinal cord with multiple small sympathetic branches protruding from the spinal cord to the target organs. Nerve fibres in the PSNS are primarily routed from the brain stem out through the ten cranial nerves, of which some pass down the neck and subsequently branch to different target organs. The 9^{th} cranial nerve (the glossopharangeal nerve) and the 10^{th} cranial nerve (the vagus nerve) are some of the most notable PSNS branches. These routings are accurate for the large majority of SNS fibres with only a few existing outside of spinal cord, some PSNS nerve fibres are routed through the spinal cord, especially those associated with the function of organs anatomically found in the pelvic region, e.g. bladder, lower gut, reproductive organs, etc.

The PNS is essentially a set of nerves that connect the CNS to every other bodily function/body part/portion (e.g. muscles, organs, cells) of the subject. Nerves serve as a conduit for transmission of neural impulses or signals to/from the CNS. For example, SoNS nerves that transmit neural impulses, signals or information from the CNS are called efferent nerves (e.g. motor nerves), while other SoNS nerves that transmit neural impulses, signals or information from one or more parts/portions of the body of the subject to the CNS are called afferent nerves (e.g. sensory nerves). Some nerves in the SoNS may have both efferent and afferent functionality and may be called mixed nerves.

In essence, the nervous system is made up of a set of nerves in which each nerve is made up of a plurality of neurons or a bundle of neurons that receive or transmit such as neural impulses or signals. A neuron has a special cellular structure that allows a nerve to send and propagate neural information rapidly and precisely to other cells, bodily functions or body parts/portions in the body of the subject. For example, the neurons in a nerve include long structures called axons that allow them to send neural impulses or signals in the form of an electrochemical gradient, also known as neural activity. A neuronal population may comprise or represent one or more neurons clustered in a location or a target site on one or more nerves of a subject.

Essentially, neural activity may comprise or represent any electrical, mechanical, chemical and or temporal activity present in the one or more neurons (or the neuronal population), which often make up one or more nerves or section(s) of neural tissue. Neural activity may convey information associated with, by way of example only but not limited to, the body of a subject and/or information about the environment affecting the body of a subject. The information conveyed by neural activity may include data representative of neural data, neural information, neural intent, end effect, tissue state, body state, neural state or state of the body, and/or or any other data, variable or information representative of the information carried or contained in neural activity and interpreted and/or passed by neurons or neuronal populations to the body of the subject. For example, neural data may include any data that is representative of the information or data that is contained or conveyed by neural activity of one or more neurons or a neuronal population. The neural data may include, by way of example only but is not limited to, data representative of estimates of one or more bodily variable(s) associated with the corresponding neural activity, or any other data, variable or information representative of the information carried or contained or conveyed by neural activity.

This information may be represented in an information theoretic point of view as one or more variables associated with the body, which are referred to herein as bodily variable(s). A bodily variable comprises or represents an end effect or tissue state describing a state of some portion of the body, including implanted or wearable medical devices. The bodily variable may itself be classified as a state, sensory, control or other variable based on the role or function of this information and the use of it by the body. Bodily variables can be transmitted to or from the CNS via neural activity in portions of the nervous system. One or more instances of neural activity at one or more neural locations can be said to be an encoding of one or more bodily variables, portions thereof and/or combinations thereof. For example, neural activity of one or more neurons of nerve(s) may be generated or modulated by part of the body to encode one or more bodily variables for reception by other parts of the body, which decode the neural activity to gain access to the bodily variable, portions thereof and/or combinations thereof. Both encoding and decoding of bodily variables can be performed by the CNS and/or bodily tissues therefore facilitating transmission of information around the body of a subject. Bodily variables can be afferent signals transmitted towards the CNS for provision of information regarding the state of bodily variables or efferent signals transmitted away from the CNS for modifying a bodily variable at an end effector organ or tissue.

The values of a group of one or more bodily variables is referred to herein as a bodily state. The bodily state of a subject is the values at a specific time of a collection of one or more relevant bodily variables.

Examples of bodily variables in the organ systems of the body, and often encoded in the ANS, could include parameters such as, by way of example only but is not limited to, current heart rate or blood pressure, current breathing rate, current blood oxygenation, instructions regarding heart pacing, instructions regarding blood vessel dilation or constriction for changing blood pressure. It is appreciated that bodily variables could be either the raw encodings or combinations of these, for instance bodily variables could include current activity of a whole organ or organ construct or measurements of whole bodily functions or actions such as hard breathing, walking, exercising, running etc; each of which it is appreciated could be described as a combination of multiple more fine grained bodily variables. In the ANS, each instance of a bodily variable may be associated with a modified organ function, modifying an organ function, or modifying a bodily function (e.g. one or more bodily variable(s) or the state of an organ or tissue). In other examples, a bodily variable may be associated with any activity in the ANS such as, by way of example only but is not limited to, organ measurement and/or modification of activity.

Although several examples of bodily variables have been described, this is for simplicity and by way of example only, it is to be appreciated by the skilled person that the present disclosure is not so limited and that there are a plurality of bodily variables that may be generated by the body of a subject and which may be sent between parts of the body or around the body as neural activity. Although neural activity may encode one or more bodily variables, portions thereof and/or combinations thereof, it is to be appreciated by the skilled person that one or more bodily variables of a subject may be measurable, derivable, and/or calculated based on sensor data from sensors capable of detecting and/or making measurements associated with such bodily variables of the subject. It is also to be appreciated by the skilled person that a bodily variable is a direct measurement of any one parameter and could be represented as a generalised parameter of activity or function in an area. This would include bodily variables such as mental states which can not be easily related to low level function such as, experiencing depression, having an epileptic fit, experiencing anxiety, having a migraine.

Although the term bodily variable is described and used herein, this is by way of example only and the present disclosure is not so limited, it is to be appreciated by the skilled person that other equivalent terms from one or more other fields (e.g. medical fields, pharmaceutical fields, biomedical fields, clinicians, biomarker fields, genomics fields, medical engineering fields) may be used in place of the term bodily variable, or used interchangeably or even in conjunction with the term bodily variable, including, by way of example only but is not limited to, one or more of the following terms or fields: vital sign(s), which is often used by clinicians to describe parameters they use for patient monitoring, such as by way of example only but is not limited to, ECG, heart rate, pulse, blood pressure, body temperature, respiratory rate, pain, menstrual cycle, heart rate variation, pulse oximetry, blood glucose, gait speed, etc.; biomarker, which may be used by biologists to describe, by way of example only but is not limited to, protein levels, or measurable indicator of some biological state or condition etc., this term has been further adopted by the Deep Brain Stimulation & Spinal Cord Stimulation clinical fields to refer to recordings of brain wave state or other neural events as well as measurement of environmental conditions including, but not limited to, motion; physiological variable/physiological data, which may often be used by scientists to describe things like ECG, heart rate, blood glucose, and/or blood pressure and the like, this term is also used by Data Sciences International who make implants for recording physiological variables such as ECG, heart-rate, blood pressure, blood glucose, etc.; one or more biosignals, which is often used by medical engineers to describe a signal recording coming from a biological system such as ECoG, ECG, EKG; any information, parameter metric about a subject in, by way of example only but not limited to, the genetic fields including, by way of example only but not limited to, genomic information, epigenetics, phenotype, genotype, other “omics” which can include, by way of example only but is not limited to, transcriptomics, proteomics and metabolomics, microbiomics, and/or other omics related fields and the like; and/or any other term describing a number, metric, state, variable or information associated with the whole body of a subject, any part and/or subpart of the body of the subject and the like.

Although examples of bodily variables are given herein, this is by way of example only and the description is not so limited, it is to be appreciated by the skilled person that the list of bodily variables is extremely large because a bodily variable may be, by way of example only but is not limited to, any number, parameter, metric, variable or information describing some state of the whole body of a subject, any portion, part and/or subpart of the body of the subject and that a bodily variable may be based on, or derived from, one or more combinations of one or more bodily variables or other bodily variables and the like. For example it is appreciated that bodily variables measured at a neurological level, biomarker level, cellular level, and/or tissue level, could combine to form bodily variables observed at a whole system state level such as regarding the vital signs of a subject; physiological meta data of a subject; sensor data representative of one or more bodily variables describing something about the body, parts of the body, or whole body of the subject; state, motion, or output of the body, part of subpart of the body of a subject and the like; modifications thereof, and/or combinations thereof and/or as herein described. Hence it is appreciated that, one or more bodily variables described at one or more higher levels of granularity may be based on a combination of one or more bodily variables described at one or more lower levels of granularity.

Although it is possible to tap into the one or more neuronal population(s) thereby effecting a direct linkage to the nervous system of a subject, there have been problems in capturing and interpreting bodily variable(s) from the neural activity generated by the neuronal population(s) and/or providing or applying neural stimulus signal(s) in order to evoke targeted responses in the form of neural activity in neuronal populations which is equivalent to or directly representing a bodily variable from device(s) to the nervous system of the subject. The bodily variable(s) may be naturally represented by neural activity associated with extremely short electrical pulses from multiple neurons. The neural activity may be received by one or more neural receivers adjacent one or more neurons or neuronal population(s) as neurological signals. These neurological signals may be sampled in which the neurological signal sampling typically provides an information rich dataset that is inordinately large, unwieldy to process, and is usually subject/experiment specific. This has led to attempts at understanding neurological signal(s) by extracting several key features thought to be representative of its information content such as bodily variable(s) encoded as neural activity.

Herein we will refer to samples or ensembles of samples of neural activity as neural biomarkers. A neural biomarker is an objective measurement of a bodily variable, including: biological processes; pathogenic processes; and/or pharmacologic responses to a therapeutic intervention, observed by monitoring one or several neural populations. Wherein neural biomarkers can represent objective indications of medical state. Neural biomarkers can be measured as features, in isolation, or linear or non-linear combinations of features, of the acquired neural population activity, which may be calculated by processing the signals, or learnt by one or more machine learning means, where this learning may be performed by a machine learning processor, either continually or in batch. One or more machine learning models running on a machine learning processor may calculate neural biomarkers having been trained on data from the nervous system of one or more patients, or from a single patient over multiple time periods, or any synthetic or simulated or biological source of neural data or activity. It is appreciated by a professional, skilled in the art, that the learnt neural biomarkers may then be used as time and or subject invariant stationary representations of the activity of the nervous system across a population of patients with the same indication, or for a single patient, or as a representation of the disease when observed in any synthetic or simulated or biological source of neural data or activity. Thus, a neural biomarker represents repeatable features from which the current neural activity can be understood as an indicator of a particular disease state or other physiological state and hence could be used as a basis for treatment decisions, therapeutic design or screening or itself be considered a useful target for direct or indirect modulation by neural, therapeutic or other means.

There is a desire for a system capable of capturing and/or interpreting bodily variable(s) encoded as neural activity, forming an accurate estimate of one or more bodily variable(s) associated with cardiac function and performing closed loop control associated with one or more cardiac functions.

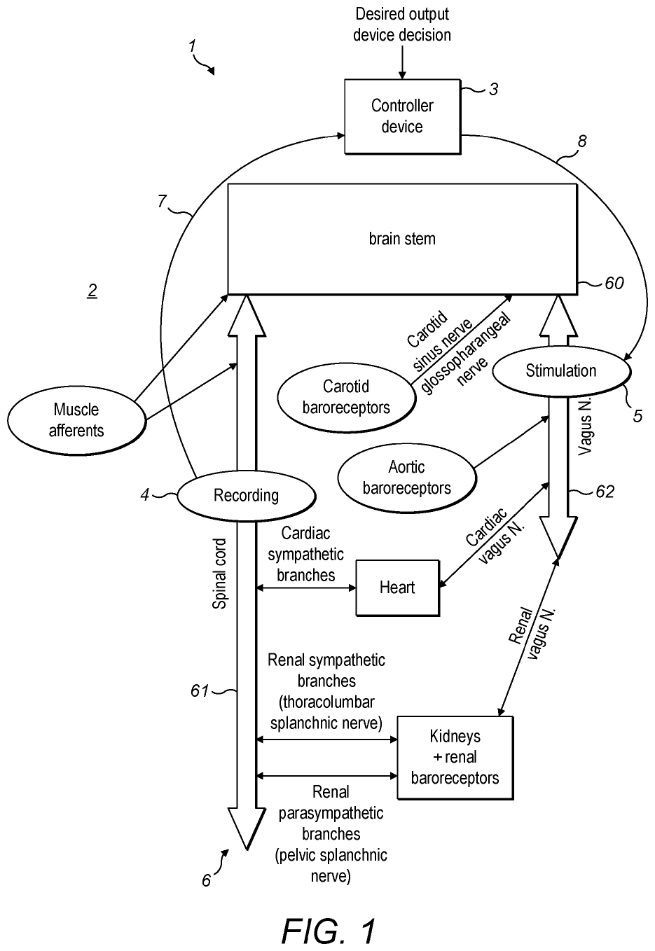

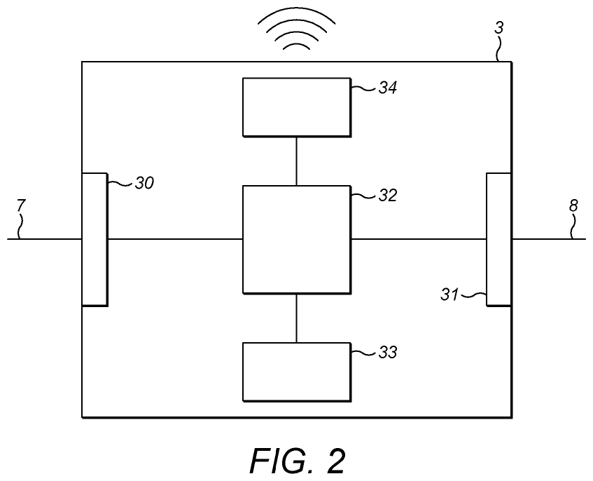

FIG. 1 shows a schematic diagram of a first example of a closed loop cardiac control system 1 according to a first embodiment. FIG. 2 shows a controller part of the closed loop cardiac control system 1 in more detail.

As shown in FIG. 1, in the illustrated first embodiment the closed loop cardiac control system 1 is embedded in a body of a human or animal subject 2. The closed loop cardiac control system 1 comprises a controller 3, a number of neural transducers 4, and a number of neural stimulators 5.

The neural transducers 4 and neural stimulators 5 of the closed loop cardiac control system 1 are embedded in the body of the subject 2, and arranged for interaction with a nervous system 6 of the subject 2. The controller 3 is connected to the neural transducers by embedded electrical connectors 7, and is connected to the neural stimulators 5 by embedded electrical connectors 8.

FIG. 1 includes a simplified schematic diagram of the nervous system 6 of the subject 2. It will be understood that the nervous system 6 is shown highly simplified and geometrically distorted in the figures in order to allow the figures to be readily understood and to explain the operation of the closed loop cardiac control system 1. As shown in FIG. 1, the nervous system 6 comprises the brain stem 60 and the spinal cord 61 and Vagus nerve 62, which are both connected to the brain stem 60. The spinal cord 61 and nerves connected to it are generally referred to as the sympathetic nervous system, while the Vagus nerve and nerves connected to it are generally referred to as the parasympathetic nervous system.

The simplified schematic diagram of the nervous system in FIG. 1 covers the primary aspects of the PSNS and SNS related to cardiovascular system control. Cardiovascular function, encompassing both the function of the heart organ itself and the peripheral vasculature, is neurally controlled by a complex interaction of sympathetic and parasympathetic signaling that bidirectionally connects the brain stem to the heart, renal system and vasculature. The spinal cord 61 carries sympathetic nerve fibres to the heart that when active function to increase cardiac activity including contractility and speed. Further the spinal cord 61 carries sympathetic nerve fibres to the renal system that when active function to increase water uptake and to the vasculature that when active cause vasoconstriction. The vagus nerve 62 carries parasympathetic nerve fibres to the heart that when active cause a reduction in cardiac activity including contractility and beating speed. Both the spinal cord 61 and the vagus nerve 62 carry parasympathetic nerve fibres to the renal system that when active cause decrease in water uptake and to the vasculature that when active cause vasodilation. Further, baroreceptor proteins present in the aortic wall, carotid arch and kidneys transmit pressure change information back to the brainstem 7 via nerve fibres in the vagus nerve, carotid sinus nerve and combined renal vagus nerve and pelvic splanchnic nerves respectively. These systems result in multiple control loops with differing timescales of control acting to maintain homeostasis of the cardiovascular system. For instance over a very short time scale during the instant pressure drop associated with standing up the baroreceptors in the aortic wall and carotid arch fire causing the brainstem to increase sympathetic firing to the heart organ such that cardiac contractility increases to increase the pressure to compensate for the orthostatic drop. Over a longer time scale the renal system reacts to low blood perfusion by decreasing water uptake such that blood volume increases and hence the pressure in the system increases to get more blood to the edges of the body. Overall it will be appreciated that there are multiple complex interactions of neural signaling with cardiovascular subcomponents that can maintain homeostasis.

In the illustrated example of the first embodiment the neural transducers 4 are embedded in the body of the subject 2 in or close to the spinal cord 61 and act as sensors to detect physiological activity in the nerves of the spinal cord 61 and to produce corresponding physiological signals. Specifically, the neural transducers 4 act as sensors to receive and detect efferent neural activity of at least one cardiac sympathetic nerve of the spinal cord 61 and to produce corresponding physiological signals comprising neural data signals. The neural transducers 4 produce electrical neural data signals corresponding to this detected efferent neural activity and sends the neural data signals to the controller 3 through the embedded electrical connectors 7.

In some examples the neural transducers 4 are embedded in the body of the subject 2 in or close to the spinal cord 61 at, or cranial to the T4 vertabrae of the subject 2, in other words, between the T4 vertabrae and the brain stem 60. However, this is not essential, and the neural transducers 4 may be embedded at other positions.

As shown in FIG. 2, the controller 3 comprises an input communication module 30, an output communication module 31, a processing module 32, a data store 33 and a communications module 34. The processing module 32 may comprise one or more processors.

In operation of the closed loop cardiac control system 1 the input communication module 30 receives neural data signals from the neural transducers 4 through the embedded electrical connectors 7. The received neural data signals are stored in the data store 33 and processed by the processing module 32 to provide processed neural data signals, which are then used to determine current cardiac function of the subject. The determined cardiac function may then be stored in the data store 33. The processing of the received neural data signals to provide processed neural data signals may comprise processing the neural data signals to identify one or more neural biomarkers. The processing module 32 then processes the determined current cardiac function of the subject and a predetermined desired cardiac function obtained from the data store 33, such as a desired cardiac function bodily setpoint, to determine one or more output signals which will affect the current cardiac function of the subject in a desired manner compared to the predetermined desired cardiac function. The determined output signals are then sent by the output communications module 35 to the neural stimulators 5 through the embedded electrical connectors. Cardiac function may also be referred to as cardiac activity.

The predetermined desired cardiac function may be provided to the closed loop cardiac control system 1 from an external system through the communications module 34 of the controller 3 and stored in the data store 33 for subsequent use. The predetermined desired cardiac function may be updated as necessary through the communications module 34. The communication module 34 supports wireless communication between the controller 3 of the closed loop cardiac control system 1 and external systems. Alternatively, the predetermined desired cardiac function may be determined by the controller 3 of the closed loop cardiac control system 1 itself.

The desired manner in which the determined output signal will affect the current cardiac function of the subject may vary depending upon the manner in which the predetermined desired cardiac function is defined, and the desired outcome for the subject, in any specific implementation. For example, if the predetermined desired cardiac function is defined as one or more predetermined values or ranges of values of parameters of cardiac function the determined output signal may be determined to modify one or more of the cardiac function parameters of the subject towards the corresponding predetermined values or ranges of values, or to remain within the corresponding predetermined ranges of values. In other examples, if the predetermined desired cardiac function is defined as one or more predetermined limits for parameters of cardiac function the determined output signal may be determined to modify one or more of the cardiac function parameters of the subject to prevent these limits being passed. In other examples, if the predetermined desired cardiac function is defined as a physiological response of the subject the determined output signal may be determined to modify cardiac function of the subject to produce the desired physiological response. The desired predetermined physiological response of the subject may be a measure associated with a return to healthy function of the cardiovascular system. For example, the desired predetermined physiological response of the subject may be one or more of a reduction in mean blood pressure, a reduction in at least one component of blood pressure, an increase in ejection fraction, and/or an increase in pulse wave velocity. The examples identified above are not intended to be exhaustive, and alternative arrangements may be used.

In some examples the predetermined desired cardiac function may be defined as a defined operating range with predetermined limits. In such examples the predetermined limits for the specific subject may be set by the controller 3. The controller may, for example base the predetermined limits on an analysis of cardiac function of the subject over time, such an analysis may, for example, be based on the processed neural data signals using at least one model.

In the illustrated first embodiment the neural stimulators 5 are embedded in the body of the subject 2 in or close to the nerves of the Vagus nerve 62 and act as stimulators to provide neural stimulation to the nerves of the Vagus nerve 62. Specifically, the neural stimulators 5 act as stimulators to provide neural stimulation to modulate efferent neural activity of at least one cardiac parasympathetic nerve of the Vagus nerve 62 to produce a physiological response in the subject.

When the neural stimulators 5 receive the output signals from the controller 3 through the embedded electrical connectors 8 the neural stimulators 5 provide neural stimulation to the at least one cardiac parasympathetic nerve of the Vagus nerve 62 based on the received output signals. This neural stimulation of the at least one cardiac parasympathetic nerve of the Vagus nerve 62 modulates the natural neural activity on the at least one cardiac parasympathetic nerve to produce a physiological response in the subject, and so affects and modifies the cardiac activity of the subject 2.

In the illustrated first embodiment the processing module 32 of the controller 3 uses one or models, such as machine learning (ML) models, to determine the current cardiac function of the subject from the received neural data signals and/or to determine the output signals. In the example described above a two-stage process is used in which one or more first ML models may be used to determine the current cardiac function of the subject from the received neural data signals, and one or more second ML models may be used to determine the output signals from the determined current cardiac activity of the subject and a predetermined desired cardiac activity. In other examples a single stage process may be used in which one or more ML models are used to determine the output signals directly from the received neural data signals and a predetermined physiological response, such as a desired cardiac function

In some examples where the neural data signals relate to physiological activity of sympathetic cardiac nerves the processed neural data signals may be used to inform one or more cardiovascular models of the sympathetic drive of the cardiac system, for example to determine at least in part the current cardiac function of the subject. These cardiovascular models of the sympathetic drive of the cardiac system may be ML models.

In some examples where the neural data signals relate to physiological activity of sympathetic renal nerves the processed neural data signals may be used to inform one or more cardiovascular models of the sympathetic drive of the renal system, for example to determine at least in part the current cardiac function of the subject. These cardiovascular models of the sympathetic drive of the renal system may be ML models.

In some examples where the neural data signals relate to afferent neural activity of renal nerves the processed neural data signals may be used to inform one or more cardiovascular models of at least one of peripheral pressure, peripheral perfusion, hypertension disease progression, or renal activity, for example to determine at least in part the current cardiac function of the subject. These cardiovascular models of the sympathetic drive of the heart may be ML models

In the illustrated example of FIG. 1 the neural data signals may be processed by the controller to determine information regarding cardiac sympathetic activity of the subject. The determined cardiac sympathetic activity may then be used to determine the current cardiac function of the subject and/or to determine the output signals.

The neural stimulators 5 may comprise any device undertaking an action resulting in or modifying neural activity in a targeted area of the neural tissue of a subject. This could include, by way of example but not limited to, operating modalities such as, electrical stimulation, chemical activation, mechanical stimulation, ultrasonic stimulation, thermal stimulation and/or optogenetic stimulation. It is not necessary that all of the neural stimulators 5 are the same. In some examples the neural stimulators 5 may include neural stimulators operating using different modalities being used together. The neural stimulators 5 may be arranged to at least partially modify neural activity, to either increase, stimulate or amplify neural activity, at least in part, or decrease or inhibit neural activity, at least in part, by selection of appropriate output signals.

In examples where the neural stimulators are arranged to increase neural activity they may be arranged to provide neural stimulation which produces an applied neural signal which is additional to natural neural signals.

In examples where the neural stimulators are arranged to decrease neural activity they may be arranged to provide neural stimulation which blocks natural neural activity, either wholly or in part. In such examples the neural stimulators may be arranged to provide neural stimulation at a frequency in the range 5 kHz to 30 kHz to block natural neural activity.

It will be appreciated by a person skilled in the art that the choice of stimulation pattern applied will have differing effects on the nerve activity. Afferent and efferent fibers have different diameters and properties, and different stimulation parameters can initiate responses of different types of fibers. By changing electrode configuration, stimulation patterns and waveforms, different fibers can be targeted. Stimulation can also be used to invoke or modulate nerve activity. Low frequency stimulation or targeted patterns can induce action potentials in neurons or groups of neurons that are transmitted along the nerves and received by the end organ as natural signals. At high frequency, sub-threshold stimulation can affect the propagation speed in fibers to reduce or modulate the signal. At higher amplitudes and in an alternative pattern, stimulation can be used to exploit the known mechanisms of action potential propagation and provide a conduction block, arresting natural nerve activity. Hence it is appreciated that the choice of stimulation can be used to induce different effects in the nerve activity.

In the illustrated first example of the first embodiment the neural transducers 4 are embedded in the body of the subject 2 in or close to the spinal cord 61 and act as sensors to detect physiological activity in the nerves of the spinal cord 61 and to produce corresponding physiological signals, whereby the neural transducers 4 act as sensors to receive and detect efferent neural activity of at least one cardiac sympathetic nerve of the spinal cord 61 and to produce corresponding neural data signals. In other examples the neural transducers 4 may be embedded elsewhere in the body of the subject 2 at a location where the neural transducers 4 are able to detect efferent neural activity of at least one cardiac sympathetic nerve. For example, the neural transducers 4 may be arranged to detect efferent neural activity of at least one cardiac sympathetic nerve in the cardiac sympathetic branches of the nervous system extending between the spinal cord 61 and the heart.

Similarly, in the illustrated first example of the first embodiment the neural stimulators 5 are embedded in the body of the subject 2 in or close to Vagus nerve 62 and act as stimulators to provide neural stimulation to nerves of the Vagus nerve 62, whereby the neural stimulators 5 act as stimulators to provide neural stimulation to modulate efferent neural activity of at least one cardiac parasympathetic nerve of the Vagus nerve 62. In other examples the neural stimulators 5 may be embedded elsewhere in the body of the subject 2 at a location where the neural stimulators 5 are able to provide neural stimulation to at least one cardiac parasympathetic nerve. For example, the neural stimulators 5 may be arranged to provide neural stimulation to at least one cardiac parasympathetic nerve in the cardiac branch of the Vagus nerve.

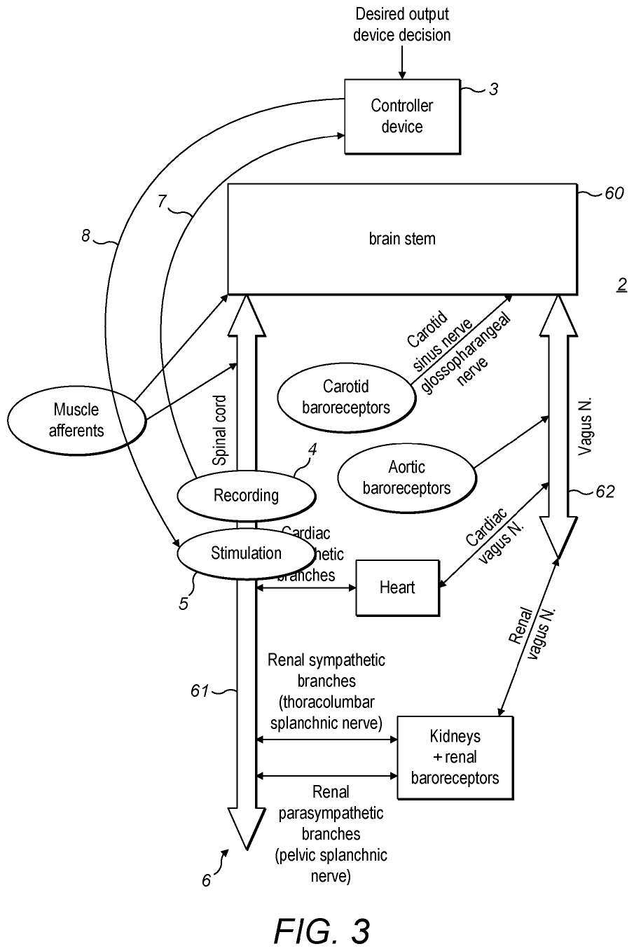

FIG. 3 shows a schematic diagram of a second example of the closed loop cardiac control system 1 according to the first embodiment.

As shown in FIG. 3, in the illustrated second example of the first embodiment the closed loop cardiac control system 1 is embedded in a body of a human or animal subject 2. The closed loop cardiac control system 1 in the illustrated second example is the same as in the first example, but is differently arranged with the neural stimulators 5 positioned at differently locations in the nervous system 6 of the subject 2.

In the illustrated second example of the first embodiment the neural transducers 4 are embedded in the body of the subject 2 in or close to the spinal cord 61 and act as sensors to detect physiological activity in the nerves of the spinal cord 61 and to produce corresponding physiological signals in a similar manner to the first example. Specifically, the neural transducers 4 act as sensors to receive and detect efferent neural activity of at least one cardiac sympathetic nerve of the spinal cord 61 and to produce corresponding neural data signals.

In the illustrated second example of the first embodiment the neural stimulators 5 are embedded in the body of the subject 2 in or close to the nerves of the spinal cord 61 and act as stimulators to provide neural stimulation to the nerves of the spinal cord 61. Specifically, the neural stimulators 5 act as stimulators to provide neural stimulation to modulate efferent neural activity of at least one cardiac sympathetic nerve of the spinal cord 61.

In some examples the neural stimulators 5 are embedded in the body of the subject 2 in or close to the spinal cord 61 at, or cranial to the T4 vertabrae of the subject 2, in other words, between the T4 vertabrae and the brain stem 60. However, this is not essential, and the neural stimulators 5 may be embedded at other positions.

In the illustrated second example of the first embodiment, when the neural stimulators 5 receive the output signals from the controller 3 the neural stimulators 5 provide neural stimulation to the at least one cardiac sympathetic nerve of the spinal cord 61 based on the received output signals. This neural stimulation of the at least one cardiac sympathetic nerve of the spinal cord 61 modulates the natural neural activity on the at least one cardiac sympathetic nerve, and so affects and modifies the cardiac activity of the subject 2.

In the illustrated second example of the first embodiment the neural transducers 4 are embedded in the body of the subject 2 in or close to the spinal cord 61 and act as sensors to detect physiological activity in the nerves of the spinal cord 61 and to produce corresponding physiological signals, whereby the neural transducers 4 act as sensors to receive and detect efferent neural activity of at least one cardiac sympathetic nerve of the spinal cord 61 and to produce corresponding neural data signals. In other examples the neural transducers 4 may be embedded elsewhere in the body of the subject 2 at a location where the neural transducers 4 are able to detect efferent neural activity of at least one cardiac sympathetic nerve. For example, the neural transducers 4 may be arranged to detect efferent neural activity of at least one cardiac sympathetic nerve in the cardiac sympathetic branches of the nervous system extending between the spinal cord 61 and the heart.

Similarly, in the illustrated second example of the first embodiment the neural stimulators 5 are embedded in the body of the subject 2 in or close to the spinal cord 61 and act as stimulators to provide neural stimulation to the nerves of the spinal cord 61, whereby the neural stimulators 5 act as stimulators to provide neural stimulation to modulate efferent neural activity of at least one cardiac sympathetic nerve of the spinal cord 61. In other examples the neural stimulators 5 may be embedded elsewhere in the body of the subject 2 at a location where the neural stimulators 5 are able to provide neural stimulation to at least one cardiac sympathetic nerve. For example, the neural stimulators 5 may be arranged to provide neural stimulation to at least one cardiac sympathetic nerve in the cardiac sympathetic branches of the nervous system extending between the spinal cord 61 and the heart.

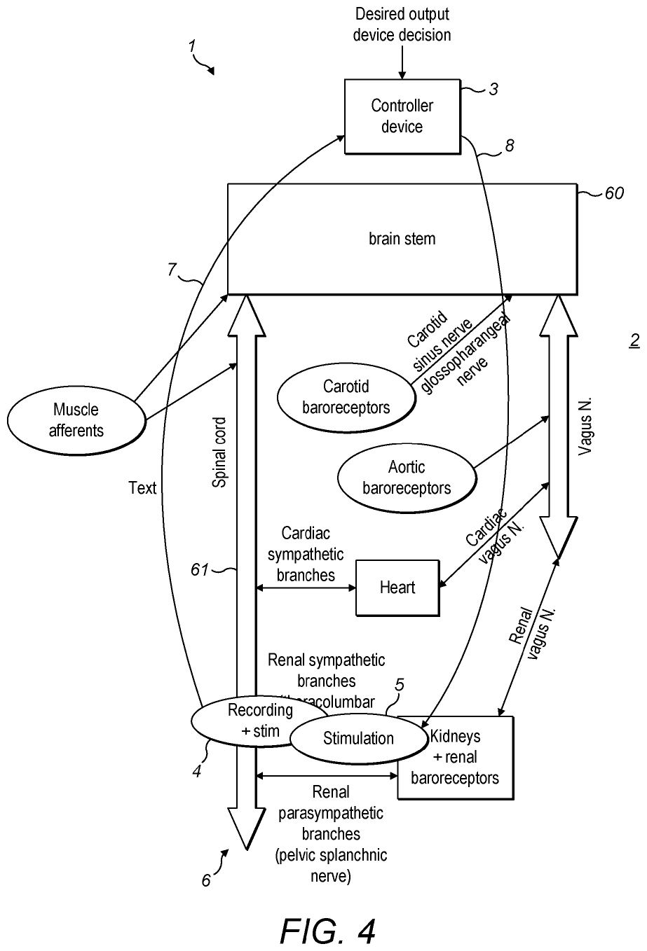

FIG. 4 shows a schematic diagram of a third example of the closed loop cardiac control system 1 according to the first embodiment.

As shown in FIG. 4, in the illustrated third example of the first embodiment the closed loop cardiac control system 1 is embedded in a body of a human or animal subject 2. The closed loop cardiac control system 1 in the illustrated third example is the same as in the first example, but is differently arranged with the neural transducers 4 and neural stimulators 5 positioned at differently locations in the nervous system 6 of the subject 2.

In the illustrated third example of the first embodiment the neural transducers 4 are embedded in the body of the subject 2 at a location where they act as sensors to receive and detect efferent neural activity of at least one renal sympathetic nerve of the subject 2 and to produce corresponding neural data signals.

In the illustrated third example of the first embodiment the neural stimulators 5 are embedded in the body of the subject 2 at a location where they act as stimulators to provide neural stimulation to modulate efferent neural activity of at least one renal sympathetic nerve of the spinal cord 61, to produce a physiological response in the subject.

In the illustrated third example of the first embodiment, when the neural stimulators 5 receive the output signals from the controller 3 the neural stimulators 5 provide neural stimulation to the at least one renal sympathetic nerve based on the received output signals. This neural stimulation of the at least one renal sympathetic nerve modulates the natural neural activity on the at least one renal sympathetic nerve, and so affects and modifies the cardiac activity of the subject 2.

In the illustrated third example of the first embodiment the neural transducers 4 may be embedded in the body of the subject 2 in or close to the spinal cord 61 and act as sensors to detect physiological activity in the nerves of the spinal cord 61 and to produce corresponding physiological signals, whereby the neural transducers 4 act as sensors to receive and detect efferent neural activity of at least one renal sympathetic nerve of the spinal cord 61 and to produce corresponding neural data signals, or may be embedded elsewhere in the body of the subject 2 at a location where the neural transducers 4 are able to detect efferent neural activity of at least one renal sympathetic nerve. For example, the neural transducers 4 may be arranged to detect efferent neural activity of at least one renal sympathetic nerve in the renal sympathetic branches of the nervous system extending between the spinal cord 61 and the kidneys.

In other examples the neural transducers 4 may be embedded in the body of the subject 2 in or close to the spinal cord 61 and act as sensors to detect physiological activity in the nerves of the spinal cord 61 and to produce corresponding physiological signals, whereby the neural transducers 4 act as sensors to receive and detect afferent neural activity of at least one renal nerve of the spinal cord 61 and to produce corresponding neural data signals, or may be embedded elsewhere in the body of the subject 2 at a location where the neural transducers 4 are able to detect afferent neural activity of at least one renal nerve. For example, the neural transducers 4 may be arranged to detect afferent neural activity of at least one renal nerve in the renal branches of the nervous system extending between the spinal cord 61 and the kidneys.

Similarly, in the illustrated third example of the first embodiment the neural stimulators 5 may be embedded in the body of the subject 2 in or close to the spinal cord 61 and act as stimulators to provide neural stimulation to the nerves of the spinal cord 61, whereby the neural stimulators 5 act as stimulators to provide neural stimulation to modulate efferent neural activity of at least one renal sympathetic nerve of the spinal cord 61, or may be embedded elsewhere in the body of the subject 2 at a location where the neural stimulators 5 are able to provide neural stimulation to at least one renal sympathetic nerve. For example, the neural stimulators 5 may be arranged to provide neural stimulation to at least one renal sympathetic nerve in the renal sympathetic branches of the nervous system extending between the spinal cord 61 and the kidneys.

In further examples of the first embodiment the neural transducers 4 may be embedded in the body of the subject 2 at a location where the neural transducers 4 are able to detect afferent neural activity of at least one renal nerve of the subject. For example, the neural transducers 4 may be arranged to detect afferent neural activity of at least one renal nerve in the spinal cord 61, or in the Vagus nerve 62, or in the pelvic nerves of the nervous system extending between the spinal cord 61 and the kidneys, or in the renal sympathetic branches of the nervous system extending between the spinal cord 61 and the kidneys.

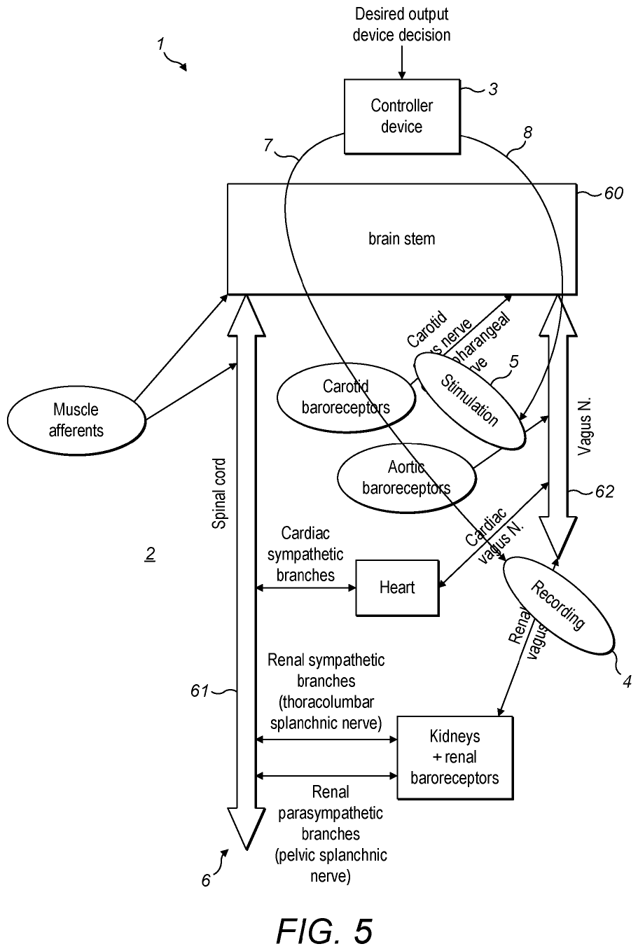

FIG. 5 shows a schematic diagram of a fourth example of the closed loop cardiac control system 1 according to the first embodiment.

As shown in FIG. 5, in the illustrated fourth example of the first embodiment the closed loop cardiac control system 1 is embedded in a body of a human or animal subject 2. The closed loop cardiac control system 1 in the illustrated fourth example is the same as in the first example, but is differently arranged with the neural transducers 4 and neural stimulators 5 positioned at differently locations in the nervous system 6 of the subject 2.

In the illustrated fourth example of the first embodiment the neural transducers 4 are embedded in the body of the subject 2 in or close to the renal branch of the Vagus nerve and act as sensors to detect physiological activity in the nerves of the renal branch of the Vagus nerve and to produce corresponding physiological signals, whereby the neural transducers 4 act as sensors to receive and detect efferent neural activity of at least one renal nerve of the renal branch of the Vagus nerve, and to produce corresponding neural data signals.

In the illustrated fourth example of the first embodiment the neural stimulators 5 are embedded in the body of the subject 2 in or close to the nerves associated with at least one baroreceptor, and specifically located in or close to the carotid sinus nerve or glossopharangeal nerve connected to the carotid baroreceptors and/or a part of the Vagus nerve connected to the aortic baroreceptors, whereby the neural stimulators 5 act as stimulators to provide neural stimulation to at least one nerve associated with the baroreceptors of the subject 2. The neural stimulators 5 may provide neural stimulation to at least one nerve associated with either or both of the carotid baroreceptors and the aortic baroreceptors. In this example the at least one nerve associated with the baroreceptors of the subject 2 may be an afferent nerve.

In the illustrated fourth example of the first embodiment, when the neural stimulators 5 receive the output signals from the controller 3 the neural stimulators 5 provide neural stimulation to the at least one nerve associated with the baroreceptors based on the received output signals. This neural stimulation of the at least one nerve associated with the baroreceptors modulates the natural neural activity on the at least one nerve associated with the baroreceptors, and so affects and modifies the cardiac activity of the subject 2.

In the illustrated fourth example of the first embodiment the neural transducers 4 are embedded in the body of the subject 2 in or close to the renal branch of the Vagus nerve and act as sensors to detect physiological activity in the nerves of the renal branch of the Vagus nerve and to produce corresponding physiological signals, whereby the neural transducers 4 act as sensors to receive and detect efferent neural activity of at least one renal nerve of the renal branch of the Vagus nerve and to produce corresponding neural data signals. In other examples the neural transducers 4 may be embedded elsewhere in the body of the subject 2 at a location where the neural transducers 4 are able to detect efferent neural activity of at least one renal nerve of the Vagus nerve. For example, the neural transducers 4 may be arranged to detect efferent neural activity of at least one renal nerve at other points in the Vagus nerve 62.

In the fourth example of the first embodiment the neural transducers 4 may be embedded in the body of the subject 2 in or close to the renal branch of the Vagus nerve, or elsewhere on the Vagus nerve, to act as sensors to detect physiological activity in the nerves of the Vagus nerve, and in particular to detect efferent neural activity of at least one renal nerve of the Vagus nerve. In other examples the neural transducers 4 may be embedded in the body of the subject 2 in or close to the renal branch of the Vagus nerve, or elsewhere on the Vagus nerve, to act as sensors to detect physiological activity in the nerves of the Vagus nerve, and in particular to detect afferent neural activity of at least one renal nerve of the Vagus nerve, and to produce corresponding neural data signals.

In the illustrated fourth example of the first embodiment the neural stimulators 5 are embedded in the body of the subject 2 in or close to the carotid sinus nerve or glossopharangeal nerve connected to the carotid baroreceptors and/or a part of the Vagus nerve connected to the aortic baroreceptors and act as stimulators to provide neural stimulation to at least one nerve associated with the baroreceptors of the subject 2. In other examples the neural stimulators 5 may be embedded elsewhere in the body of the subject 2 at a location where the neural stimulators 5 are able to provide neural stimulation to at least one to at least one nerve associated with the baroreceptors of the subject 2. For example, the neural stimulators 5 may be arranged to provide neural stimulation to at least one renal parasympathetic nerve fibre in the vagus or pelvic nerves.

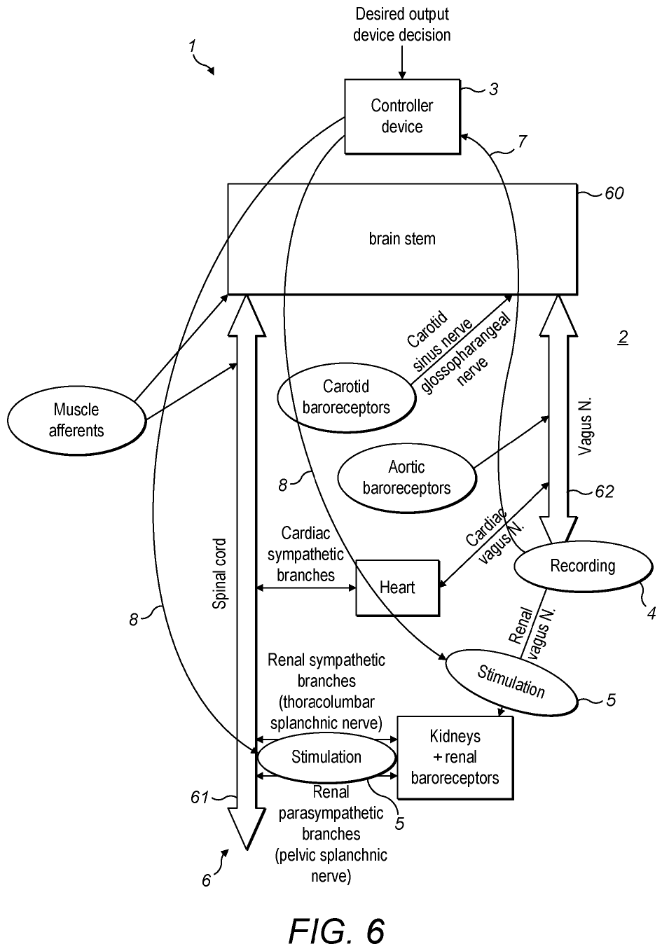

FIG. 6 shows a schematic diagram of a fifth example of the closed loop cardiac control system 1 according to the first embodiment.

As shown in FIG. 6, in the illustrated fifth example of the first embodiment the closed loop cardiac control system 1 is embedded in a body of a human or animal subject 2. The closed loop cardiac control system 1 in the illustrated fifth example is the same as in the first example, but is differently arranged with the neural transducers 4 and neural stimulators 5 positioned at differently locations in the nervous system 6 of the subject 2.

In the illustrated fifth example of the first embodiment the neural transducers 4 are embedded in the body of the subject 2 in or close to the renal branch of the Vagus nerve and act as sensors to detect physiological activity in the afferent nerves of the renal branch of the Vagus nerve and to produce corresponding physiological signals, whereby the neural transducers 4 act as sensors to receive and detect afferent neural activity of at least one renal nerve of the renal branch of the Vagus nerve and to produce corresponding neural data signals. The neural transducers 4 are arranged to detect afferent neural activity in the Vagus nerve and to produce physiological signals comprising neural data signals corresponding to this detected neural activity, and accordingly the controller will process these neural data signals to determine information regarding cardiac parasympathetic activity of the subject.

In the illustrated fifth example of the first embodiment the neural stimulators 5 are embedded in the body of the subject 2 in or close to the renal Vagus nerve and/or the renal sympathetic branches of the nervous system extending between the spinal cord and the kidneys (the thoracolumbar splachnic nerve) and/or the renal parasympathetic branches of the nervous system extending between the spinal cord and the kidneys (the pelvic splanchnic nerve), whereby the neural stimulators 5 act as stimulators to provide neural stimulation to at least one efferent sympathetic renal nerve and/or efferent parasympathetic renal nerve. In some examples the neural stimulators 5 may provide neural stimulation to at least one efferent sympathetic renal nerve and at least one efferent parasympathetic renal nerve.

In the illustrated fifth example of the first embodiment, when the neural stimulators 5 receive the output signals from the controller 3 the neural stimulators 5 provide neural stimulation to the at least one efferent sympathetic renal nerve and the at least one efferent parasympathetic renal nerve. This neural stimulation of the at least one sympathetic renal nerve and the at least one efferent parasympathetic renal nerve modulates the natural neural activity on the at least one sympathetic renal nerve and the at least one efferent parasympathetic renal nerve, and so affects and modifies the cardiac activity of the subject 2.

In the illustrated fifth example of the first embodiment the neural transducers 4 and neural stimulators 5 may be embedded in the body of the subject 2 in or close to the renal branch of the Vagus nerve to act as sensors to detect physiological activity in the nerves of the renal branch of the Vagus nerve or to stimulate the nerves of the renal branch of the Vagus nerve respectively. In other examples the neural transducers 4 and neural stimulators 5 may be embedded elsewhere in the body of the subject 2 at a location where the neural transducers 4 and neural stimulators 5 are able to interact with at least one renal nerve of the Vagus nerve. For example, the neural transducers 4 and neural stimulators 5 may be respectively arranged to detect afferent neural activity and modulate efferent neural activity of at least one renal nerve at other points in the Vagus nerve 62.

In an alternative example the neural transducers 4 may be embedded in the body of the subject 2 in or close to the cardiac branch of the Vagus nerve to act as sensors to detect physiological activity in the nerves of the cardiac branch of the Vagus nerve. In such examples the system may also comprise further neural transducers 4 arranged to detect neural activity in the nervous system of the subject at one or more of carotid baroreceptors, aortic baroreceptors, renal afferent nerves, and muscular afferent nerves.

The illustrated examples of the first embodiment described above have neural transducers 4 and neural stimulators 5 arranged at a number of different locations in the nervous system of the subject 2. These different locations are not exhaustive, and are provided by way of example only. It should be understood that the different locations of the neural transducers 4 and neural stimulators 5 in the different examples may be arranged in different combinations in other examples, and may be replaced by or combined with neural transducers and/or neural stimulators arranged at other locations in the nervous system of the subject 2.

In some examples the plurality of neural transducers may arranged to detect neural activity in the nervous system of the subject at one or more of the brain stem, the upper spinal cord, the cardiac sympathetic branches, the renal sympathetic branches, the upper Vagus nerve, the cardiac branch of the Vagus nerve, the renal branch of the Vagus nerve, renal afferent nerves, muscle afferent nerves, and/or barroreceptors. The barroreceptors may comprise carotid baroreceptors and/or aortic barroreceptors. This list is provided purely by way of example and is not intended to be exhaustive. The plurality of neural transducers may be arranged to detect neural activity relating to at least one of sympathetic afferent neural signals or sympathetic efferent neural signals going to at least one of a cardiac system or a renal system of the subject

In some examples the plurality of neural stimulators may be arranged to provide neural stimulation to the nervous system of the subject at any one or more of the brain stem, the upper spinal cord, the; cardiac sympathetic branches, the renal sympathetic branches, the upper Vagus nerve, the cardiac branch of Vagus nerve, and/or the renal branch of Vagus nerve. This list is provided purely by way of example and is not intended to be exhaustive. The plurality of neural stimulators may be arranged to modify neural activity relating to at least one of sympathetic afferent neural signals or sympathetic efferent neural signals going to at least one of a cardiac system or a renal system of the subject.

In some examples the plurality of neural stimulators may be arranged to provide neural stimulation to one or more of sympathetic cardiac neural pathways in the spinal cord, sympathetic renal neural pathways in the spinal cord, parasympathetic renal neural pathways in the Vagus nerve, and/or parasympathetic renal neural pathways in the Vagus nerve.

In some examples the plurality of neural transducers may arranged to detect neural activity in the nervous system of the subject at the Vagus nerve and/or the spinal cord and the plurality of neural stimulators may be arranged to provide neural stimulation to the Vagus nerve and/or the spinal cord.

It should be understood that it is not essential that the neural transducers 4 are separate devices from the neural stimulators 5. In implementations where sensing and stimulation are both carried out at the same locations in the nervous system of the subject 2 some or all of the neural transducers 4 and neural stimulators 5 may be combined in dual purpose sensor/stimulator devices. Hence the illustrated functional separation is not indicative of physical separation. Examples of such an implementation where sensing and stimulation may be both carried out at the same location are shown in FIGS. 3, 4 and 6.

The signals may be carried between the controller 3, the neural transducers 4, and the neural stimulators 5 in any suitable manner. In the illustrated examples these signals are carried by a wired communication system in other examples a wireless communication system may be used.

In addition to the neural transducers 4 shown in FIGS. 1 to 6 the closed loop cardiac control system 1 may also receive information from sensors which are arranged to obtain bodily variable data regarding bodily variable parameter values of the subject 2, and to provide this bodily variable data to the controller 3 of the closed loop cardiac control system 1. These sensors may, for example, be wearable sensors or embedded sensors. The sensors may be arranged to communicate with the closed loop cardiac control system 1 by any suitable means, such as a wired or wireless communication system. In some examples the sensors may be comprised in the closed loop cardiac control system 1.

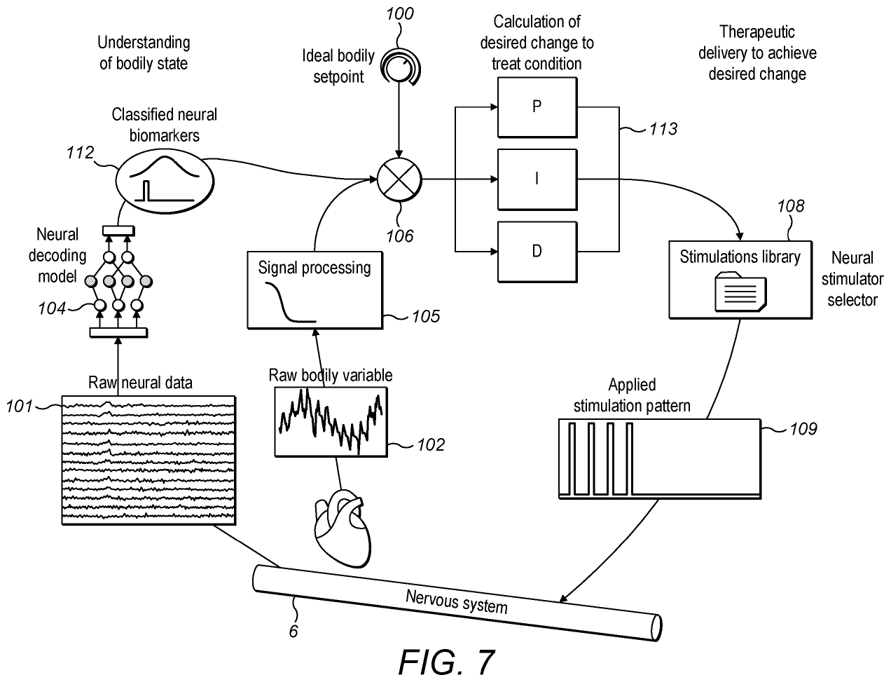

FIG. 7 shows a representation of a specific example of the data flow through the closed loop cardiac control system 1 according to the first embodiment in the examples of FIGS. 1 to 6.

In the illustrated example of FIG. 7 the closed loop cardiac control system 1 is used to provide closed loop control of a cardiac bodily state of a subject in order to bring the cardiac bodily state into, or at least closer to, agreement with a desired value of that cardiac bodily state. In order to do this an ideal cardiac bodily setpoint 100 corresponding to a desired operating point of the cardiac system of the subject, or a desired value of the cardiac bodily state being controlled, is set. This ideal bodily setpoint 100 may be fixed, or may be determined based on information about the subject. The ideal bodily setpoint 100 may be pre-set, may be determined by the controller 3, or may be provided to the cardiac control system 1 by another system, as appropriate in any specific implementation. By a bodily setpoint is meant the standard meaning in the technical field of the invention of a setpoint for a control system, and is the desired or target value for bodily variable or bodily state. Departure of the bodily variable or bodily state from its setpoint is one basis for error-controlled regulation using negative feedback for automatic control.

In some examples where the ideal bodily setpoint 100 is determined by the controller 3, the controller 3 may use at least one model, such as an ML model, to determine the desired operating point of the cardiac system of the subject. In such examples the at least one model may be constrained to keep the desired operating point of the cardiac system to be one or more predetermined values, or to be maintained not to pass one or more predetermined limits.

In the specific example of FIG. 7 the cardiac bodily state is the heart rate of the subject, and the ideal cardiac bodily set point 100 is a specific heart rate value. In some examples a desired value for the heart rate may be calculated by the controller 3 of the cardiac control system 1 based, for example, on other bodily variables, e.g., the current blood pressure or current physical activity level or overall cardiovascular health of the subject. In other examples the desired value for the setpoint of heart rate may be provided to the neural control system 1 by a clinician.

In operation of the cardiac control system 1, the neural transducers 4 obtain neural data relating to a bodily state of the subject 2 from the nervous system of the subject 2. In the illustrated example further sensors obtain bodily variable data regarding bodily variable parameter values. FIG. 7 shows illustrative examples of full spectrum multiple channel neural data 101 relating to a heart rate of the subject which may be produced by the neural transducers 4, and body variable data 102 in the form of blood pressure values, which may be produced by the additional sensors.

In the example of FIG. 7, neural data 101 and body variable data 102 is provided by the neural transducers 4 and the additional sensors to the controller 3 for processing. In the controller 3 the neural data 101 and body variable data 102 is subject to a machine learning (ML) processing. The ML processing comprises processing of the full spectrum multiple channel neural data 101 using a neural decoding machine learning (ML) model 104 to identify neural biomarkers, in this example, classified neural biomarkers 112. The example of FIG. 7 further shows signal processing 105 applied to the body variable data 102 by the controller 105. It is not essential that this signal processing 105 uses an ML model.

In the illustrated example of FIG. 7 the body variable data 102 is a blood pressure signal comprising a series of blood pressure values.

In general, the ML model is a model which has been previously generated by machine learning techniques, such as a forward pass model, and provided to the cardiac control system 1. In general machine learning will not actually be carried out by the controller 3 of the cardiac control system 1 itself, with currently available technologies such machine learning is generally to demanding of computing resources to be practically provided in an implanted device. However, it is possible that the carrying out of machine learning within the controller 3 may be practical in the future.

In the example of FIG. 7, the classified neural biomarkers 112 produced by the ML model 104 and the output data produced by the signal processing 105 are combined to determine a current cardiac bodily state, and in this example specifically the heart rate. This determined current cardiac bodily state (current heart rate) then is compared at a summing junction 106 with the ideal cardiac bodily set point (specific heart rate value) 100 to calculate a desired change in the value of the cardiac bodily state (heart rate) to bring the cardiac bodily state (heart rate) into, or at least closer to, agreement with the ideal cardiac bodily set point (specific heart rate value) 100. The summing junction 106 compares the inputs it receives of the determined cardiac bodily state (heart rate) values and the values of the ideal cardiac bodily setpoint (specific heart rate value) 100, and outputs the difference between these values. As shown in FIG. 7, the output of the summing junction 106 may be used in a PID (proportional-integral-derivative) calculation by a PID controller 113, as commonly used in feedback control systems. PID controllers are well known to the skilled person, so it is not necessary to describe this in detail herein. In the illustrated first example the summing junction 106 carries out a simple subtraction of its two inputs. In other examples different comparisons, such as more complex operations, may be carried out. Thus, the controller 3 makes a control decision to change the current operating point of the cardiac system of the subject towards the determined desired operating point.

In the illustrated example of FIG. 7, the desired change in the value of the bodily state produced by the summing junction 106 is subjected to data processing using a further ML model to determine a set of output signals which are neural stimulation signals required to bring about the desired change. For example, to move one or more parameters of the cardiac function towards the desired operating point of the cardiac system of the subject according to the previously made control decision. As shown in FIG. 7, the further ML model may be a simulation library selector 108 selecting as output signals an appropriate set of neural stimulation signals from a library of possible stimulation signals. The set of neural stimulation signals selected as the output signals may, for example, define which of the neural stimulators 5 are to produce neural stimulation signals, and the timing and modulation of these neural stimulation signals. FIG. 7 shows an illustrative example 109 of a set of output signals which is a pattern of applied stimulation signals comprising a set of neural stimulation signals.

In the illustrated example of FIG. 7, the determined set of neural stimulation signals is produced as output signals by the controller 3, whereby the controller 3 sends electrical neural stimulation control signals as output signals to selected ones of the neural stimulators 5 as required for the neural stimulators 5 to generate and apply the desired set of neural stimulation signals to the nervous system of the subject 2.

In some examples the controller 3 may be arranged to produce the at least one output signal using at least one model arranged determine and produce at least one output signal to bring cardiac function of the subject closer to that of a healthy subject. In such examples the ideal cardiac bodily setpoint 100 may be selected to correspond to values of a healthy subject.

In some examples the controller 3 may be arranged to identify that detected natural efferent activity on a nerve would have the effect of moving one or more parameters of the cardiac function of the subject past one or more predetermined values, such as predetermined limits, or away from a desired operating point, and to respond to this identification by determining and producing an output signal which will cause one or more of the neural stimulators to block the detected natural efferent activity on that nerve.

In some examples the controller 3 may use a body model as an ML model to combine the classified neural biomarkers 112 produced by the ML model 104 and the output data produced by the signal processing 105 to determine a current cardiac bodily state, and specifically the heart rate. A body model is an internal dynamical model of the body of the subject, which is used by the controller 3 to calculate the optimum actions for control. The body model may, for example, be a white box model, an input/output model, a state space model, or any other model of the system. The model may be used to produce an estimate or prediction of a current bodily state of a subject using a model predictive control process (MPC) process, wherein the model may comprise an updating model predictive controller. Accordingly, the estimated or predicted current state of the body may be informed by a combination of the neural biomarkers 104 produced by the ML data processing 103 and/or the data from the additional sensors. Body models and model Predictive Controllers are well known to the skilled person, so it is not necessary to describe this in detail herein.

In examples using a body model the controller 3 may use the history of past stimulation and the subsequent changes in bodily variables to update the body model.

In some examples the controller 3 may use a setpoint calculator to use the received bodily variable data and/or neural signal data to calculate an estimate of the ideal bodily setpoint 100, and to output this estimated ideal bodily setpoint 100 to the summing junction 106. The setpoint calculator may alternatively receive data to calculate the ideal bodily setpoint from other sources.

In some examples a summing junction 106 may not be used and the ideal bodily setpoint may be provided using an ML model.

In examples where the bodily state is the heart rate of the subject, the setpoint calculator may, for example, choose to reduce the bodily setpoint of heart rate when a bodily variable of blood pressure increases to dangerous levels, or is increasing dangerously rapidly.

In the illustrated example of FIG. 7, blood pressure values 102 are provided by the additional sensors. In other examples the blood pressure values may be determined from neural data obtained by the neural transducers from one or more nerves associated with baroreceptors, the blood pressure values may, for example, be determined using an ML model.

The system architecture and method of operation of the closed loop cardiac control system 1 described above enables the closed loop cardiac control system 1 to provide a control loop allowing effective closed loop control based upon real time nerve information. Further, the closed loop cardiac control system 1 can provide closed loop performance based directly on cardiac performance and/or biologically or medically relevant features derived from the received neural signals.

In examples where the locations of the neural transducers 4 and the neural stimulators 5 in the nervous system of the subject 2 are such that at least some of the neural transducers 4 may directly receive the neural stimulation signals generated by the neural stimulators 5, the controller 3 may stop the recording of neural data by the affected ones of the neural transducers 4 for the duration of the neural stimulation signals, in order to prevent cross talk between the neural stimulators 5 and the neural transducers 4 reducing the quality of the received neural data. In some examples the affected neural data may be replaced by blanket zeros during the stimulation. In some examples the neural transducers 4 may be switched off or deactivated for the duration of the neural stimulation signals.

In the examples the controller 3 provides a single output of nerve stimulation signals to control a single bodily variable. In other examples the controller 3 may provide multiple sets of nerve stimulation signals to control multiple bodily variables.

In the example of FIG. 7 described above the controller 3 uses the neural data from the neural transducers 4 and a blood pressure signal comprising blood pressure values from a further sensor arranged to obtain bodily variable data regarding bodily variable parameter values, and specifically blood pressure values, to determine the output signals. In some examples the blood pressure signal identifies blood pressure of the subject and comprises at least one of, Systolic pressure, Diastolic pressure, peak pressure, Orthostatic drop, mean blood pressure, Ejection Time, Pulse Wave Velocity, or other relevant features derived from the signal identifying the blood pressure in the cardiac system.

In other examples the controller 3 may use the neural data from the neural transducers 4 and other bodily variable parameter values from suitable further sensors. In some examples the further sensors may be arranged to provide bodily variable parameter values in the form of a heart signal identifying electrical activity of the subjects heart, and the controller may be arranged to process this heart signal together with the neural data to determine the one or more output signals using a model, such as an ML model. In such examples the heart signal may identify electrical activity of the subjects heart comprising at least one of Heart Rate, Heart Rate Variability, P wave shape, T wave duration, T wave amplitude, J point, ST elevation, U wave, R-R interval, signal period, frequency profile, amplitude, or other relevant features derived from the signal identifying electrical activity of the heart. This list is not intended to be exhaustive and is provided by way of example only.

In some examples the controller 3 may be arranged to receive the neural data from the neural transducers 4 and both the blood pressure signal and the heart signal from further sensors, and to process these to determine the one or more output signals using a model, such as an ML model.

In some examples the controller 3 may be arranged to receive both the blood pressure signal and the heart signal from further sensors, and to process these together to identify cardiac activity of the subject comprising at least one of: Q-A interval, Baroreceptor Sensitivity, Volumetric Cardiac Output or other relevant features derived from joint cross-analysis of the heart electrical signal and blood pressure signal.

The controller 3 may be arranged to produce the output signals substantially continuously based on the received neural data. In some examples the controller 3 may be arranged to provide at least one output signal in response to identification of a predetermined event. The predetermined event may, for example, be a neural event identified based on the received neural data. In other examples the predetermined event may, for example, be a non-neural event identified from other non-neural received data. The non-neural received data may in some examples be data from the further sensors.

In examples where the predetermined event is a neural event, the predetermined event may for example be one or more of baroreceptor firing indicative of blood pressure changes, renal afferent firing indicative of low blood perfusion, and sympathetic firing indicative of cardiac upregulation. In examples where the predetermined event is a non-neural event, the predetermined event may for example be one or more of heart rate too high, heart rate too low, blood glucose too high, blood glucose too low. These lists are exemplary only and are not intended to be exhaustive.

In some examples the controller 3 may comprise, in addition to the model or models used to determine at least one output signal to bring cardiac function of the subject closer to that of a healthy subject, at least one further model arranged to determine at least one output signal to bring the function of another organ closer to that of a healthy subject.

In some examples the controller 3 may be arranged to produce at least one output signal in response to identification of a blood pressure of the subject exceeding a predetermined threshold value.

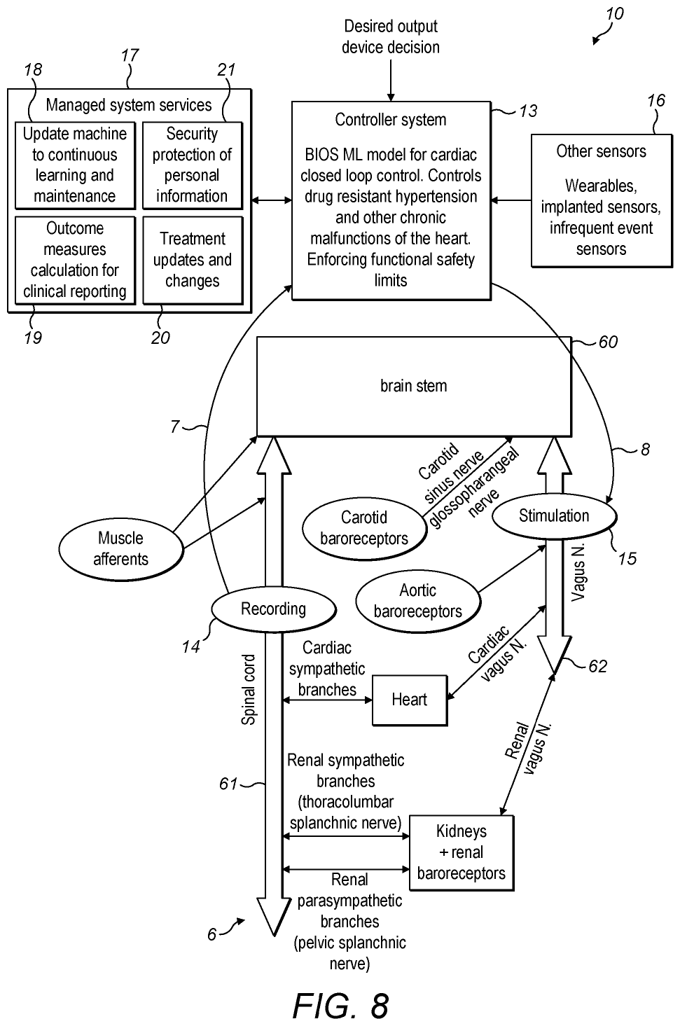

FIG. 8 shows a schematic diagram of a closed loop cardiac control system 10 according to a second embodiment.

The closed loop cardiac control system 10 according to the second embodiment is similar to the system 1 of the first embodiment, and comprises a controller 13, a number of neural transducers 14, and a number of neural stimulators 15. These operate in a similar manner to the corresponding parts of the closed loop cardiac control system 1 according to the first embodiment.

In the illustrated example of the closed loop cardiac control system 10 according to the second embodiment of FIG. 8 the neural transducers 14 and neural stimulators 15 are arranged in corresponding locations to the neural transducers 4 and neural stimulators 5 of the first example of the first embodiment of FIG. 1. These may be arranged at other locations in a similar manner to the first embodiment described above.

The closed loop cardiac control system 10 according to the second embodiment further comprises a number of further sensors 16, in addition to the neural transducers 14. The sensors 16 may be arranged to detect data regarding bodily variables of the subject 2 and to provide this body variable data to the controller 13, typically through the communications module 34. The body variable data may, for example, be values of blood pressure, heart rate, or other body variable parameters. The sensors 16 may also comprise sensors to identify events which may affect the body of the subject 2, such as high levels of physical activity. The sensors 16 may also comprise infrequent event sensors detecting discrete events that are relevant in understanding the state of the patient, these could include by way of example but not limited to transitioning to sleeping/waking taking of medication, entering a warmer or colder temperature environment, etc. The sensors 16 may include embedded sensors, wearable sensors, and sensors comprised in wearable devices. The sensors 16 may comprise one or more electrical sensors arranged to sense electrical activity of the subjects heart and to generate a heart signal. The sensors 16 may comprise one or more blood pressure sensors arranged to sense a blood pressure of the subject and to generate a blood pressure signal.

In the closed loop cardiac control system 10 according to the second embodiment the controller 13 may use the body variable data and/or other data received from the sensors 16 to determine the current cardiac activity of the subject, in combination with the neural data from the neural transducers 14.

The closed loop cardiac control system 10 according to the second embodiment is arranged to communicate with other external systems 17. The controller 13 can communicate with these external systems 17 through the communications module 34.

The external systems 17 may provide a number of different functions to support the closed loop cardiac control system 10. The external systems 17 may, for example, be provided by a network of cloud servers.

The external systems 17 may comprise an update machine 18. The closed loop cardiac control system 10 may send data regarding the operation of the closed loop cardiac control system 10 and data regarding the subject 2 to the update machine. The update machine 18 may include machine learning data processing retraining systems and machine learning controller retraining systems which retrain machine learning models using high powered computers, such as cloud computers, based on the data provided by cardiac control system 10. This retraining may be carried out with input from machine learning researchers developing new and improved machine learning models. This retraining may also be based on data received from other cardiac control systems additional to the cardiac control system 10.

The update machine 18 generates updated machine learning models by machine learning training using the neural data received from the cardiac control system 10, and possibly also other data from other sources. The update machine 18 may periodically, or as necessary, send updated machine learning models or machine learning model updates to the cardiac control system 10. The cardiac control system 10 receives the updated machine learning models or machine learning model updates from the update machine 18, or other update machines, and uses these to update or replace the machine learning model or models used by the controller 13, as required.

The updates to the machine learning models may be calculated based on the received neural data, calculated neural biomarkers, output signals, recorded neural data, data from other sensors, and/or data representing bodily state and/or any other data saved by the neural control system. The updates may be generated based on data recorded by the cardiac control system 10 during specific periods of guided activity during rehabilitation or recalibration periods. The update machine 18 may be provided by an automated cloud system.

In some examples the update machine 18 may be a manual connection over a local wired or wireless connection. In some examples the updates may be automatically calculated by one or more machine learning systems for calculating long term treatment. In some examples the updates may be chosen by a treating clinician.

The interaction of the cardiac control system 10 with the update machine 18 described above enables the cardiac control system 10 to be provided with an external control loop allowing updating of the machine models used based upon data obtained from the subject and the performance of the cardiac control system 10. This external control loop is relatively low speed, or relatively high delay/latency compared to the operation of the cardiac control system 10. The external control loop enables performance of the machine learning models, and thus the cardiac control system 10, to be improved over time as more data is gathered by the cardiac control system 10, and other cardiac control systems.

In some examples the update machine 18 may be arranged to update the cardiac control system 10 to change which cardiac function parameters of the subject the cardiac control system 10 controls.

It should be appreciated that the machine learning model data processing set out above is described by way of example only and that any number of machine learning models could be used, utilizing many types of architecture to process data. In some examples multiple machine learning models may be run simultaneously in parallel with a majority vote, state space model or other decision making module deciding on the device output action based on the outputs of multiple ones of the machine learning models. The different machine learning models may be of different types and operate over different timescales.

The external systems 17 may comprise a clinician reporting system 19. The closed loop cardiac control system 10 may send data regarding the operation of the closed loop cardiac control system 10 and data regarding the subject 2 to the clinician reporting system 19. The clinician reporting system 19 may use this data to calculate subject outcome measures for the cardiac control system 10. The subject outcome measures may provide metrics or other indicators regarding the cardiac performance and/or health of the subject and how much beneficial effect is being achieved by the cardiac control system 10. The subject outcome measures may be reported to a clinician, or other supervisor, such as a clinician responsible for treatment of the subject 2, to be taken into account when considering any further action to be taken regarding the subject.

The external systems 17 may comprise a treatment information system 20. The treatment information system 20 is arranged to inform the cardiac control system 10 regarding treatments being provided to the subject 2. The treatment information system 20 may inform the cardiac control system 10 regarding treatments being provided to the subject 2 when the cardiac control system 10 begins operation so that the cardiac control system 10 may take any effects of the treatments on the cardiac function of the subject into account. Further, the treatment information system 20 may inform the cardiac control system 10 of updates or changes to treatments being provided to the subject 2. If these updates or changes to treatments affect the cardiac function of the subject 2 the cardiac control system 10 may make appropriate changes to take these into account.

The external systems 17 may comprise a security system 21. The security system 21 may control access to and protect personal information regarding the subject 2 which has been passed to the external systems 17 by the cardiac control system 10, or which is otherwise held by the external systems 17. Further, the security system 21 may control the sending of information, such as updates, to the cardiac control system 10 by the external systems 17 to prevent unauthorized or malicious activity.

In some examples the different parts 18 to 21 of the external systems 17 may be a single integrated system, in other examples they may be separate systems. In some examples only some of the parts 18 to 21 of the external systems 17 may be provided.

In some examples of the closed loop cardiac control systems 1 and 10 the controller 3 or 13 may be arranged to receive at least one signal associated with a blood pressure rise and fall of the subject. One example of this is shown in FIG. 7 where a blood pressure signal comprising a series of blood pressure values is received by the controller 3. As discussed above with reference to FIG. 7, this blood pressure signal may be used by the controller to determine the at least one output signal sent to the neural stimulators.

In a third embodiment, a closed loop cardiac control system is similar to the systems 1 and 10 of the first and second embodiments, and comprises a controller a number of neural transducers and a number of neural stimulators. These operate in a similar manner to the corresponding parts of the closed loop cardiac control systems 1 and according to the first and second embodiments.

In the third embodiment the one or more neural transducers are arranged to act as sensors to receive and detect efferent neural activity travelling to the heart of the subject. Accordingly, the one or more neural transducers may be arranged to receive and detect parasympathetic efferent neural cardiac activity, and so act as at least one parasympathetic efferent cardiac activity sensor 205, and/or to receive and detect sympathetic efferent neural cardiac activity, and so act as at least one sympathetic efferent cardiac activity sensor 206. For example, the neural transducers may be arranged similarly to the first and second examples of the first embodiment shown in FIGS. 1 and 2. The neural transducers acting as at least one parasympathetic efferent cardiac activity sensor 205 and/or at least one sympathetic efferent cardiac activity sensor 206 produce at least one neural data signal associated with the detected efferent neural activity and send this at least one neural signal to the controller for processing.

The controller is arranged to process the received at least one neural data signal to determine and register the timing and magnitude of the sensed natural efferent neural signals to the heart. Similarly to the previous embodiments the controller may use one or more models, which may be ML models to carry out this processing.

In the third embodiment at least one further sensor is arranged to produces at least one signal associated with the rise and fall of the blood pressure of the subject. The at least one further sensor may also be provided by one or more neural transducers. These neural transducers may be arranged to receive and detect afferent neural activity coming from baroreceptors of the subject, and in particular afferent neural activity coming from renal baroreceptors or cardiac baroreceptors of the subject. For example, the neural transducers may be embedded in the body of the subject in or close to the carotid sinus nerve, the glossopharangeal nerve connected to the carotid baroreceptors, a part of the Vagus nerve connected to the aortic baroreceptors, or the pelvic nerves. Accordingly, the neural transducers can provide at least one neural sensor 201 for a renal afferent baroreceptor and/or at least one neural sensor 202 for a cardiac afferent baroreceptor, to produce at least one neural data signal associated with the rise and fall of the blood pressure of the subject, and send this at least one neural data signal associated with the rise and fall of the blood pressure to the controller for processing. Alternatively, or additionally, in the third embodiment at least one blood pressure sensor 203 is arranged to produces at least one blood pressure signal associated with the rise and fall of the blood pressure of the subject, and send this to the controller for processing.

The controller is arranged to process the received at least one neural data signal associated with the rise and fall of the blood pressure and/or the at least one blood pressure signal to determine and register the timing and magnitude of changes in the blood pressure of the subject. Similarly to the previous embodiments the controller may use one or more models, which may be ML models to carry out this processing.

In some examples the different received signals are all sampled at a frequency greater than 10 Hz in order to capture the beat to beat blood pressure rise and fall and neural activity having a corresponding timescale.

The controller is arranged to associate the timing of the natural efferent neural signals to the heart with respect to the timing of any blood pressure change, and to determine the relationship between the timing of the natural efferent neural signals to the heart and the timing of any corresponding blood pressure change. The controller is further arranged to associate the magnitude of the natural efferent neural signals, or a calculated efferent cardiac response, with a magnitude of any corresponding blood pressure change, and to determine the relationship between the magnitude of the natural efferent neural signals and the magnitude of any corresponding blood pressure change.

Baroreceptors are natural pressure sensitive neurons that fire in response to changes in blood pressure on a beat to beat basis, that is, the baroreceptors fire and slow down over each heart beat cycle as the blood pressure rises and falls. The brainstem controls immediate efferent neural activity on both the sympathetic and parasympathetic nervous pathways to the heart in response to the received baroreceptor signaling. This natural mechanism controls physiological parameters such as orthostatic blood pressure drop. The sensitivity or responsiveness of the brainstem to second to second baroreceptor signaling is generally referred to as baroreceptor sensitivity, and is traditionally measured by the phase shift and amplitude response of the change in heart rate (measured by ECG) to beat to beat blood pressure changes and it is used to diagnose many cardiac conditions.

The determined timing and magnitude relationships between the natural efferent neural signals to the heart and the corresponding blood pressure changes may be used by the controller 3 to determine baroreceptor sensitivity or response, or may be provided to other systems for use to determine baroreceptor sensitivity or response. The determined timing and magnitude relationships and/or the determined baroreceptor sensitivity or response may be used to determine cardiac health and/or to diagnose cardiac conditions for the subject.

In the third embodiment the controller may be arranged to also use the received at least one neural signal associated with the rise and fall of the blood pressure and/or the at least one blood pressure signal to determine the at least one output signal to be sent to the neural stimulators in a corresponding manner to those described with reference to the first and second embodiments. In some examples the controller may be arranged to use the determined magnitude of changes in the blood pressure of the subject to determine the at least one output signal. In some examples the controller may be arranged to use the magnitude of the at least one blood pressure signal to determine the at least one output signal.

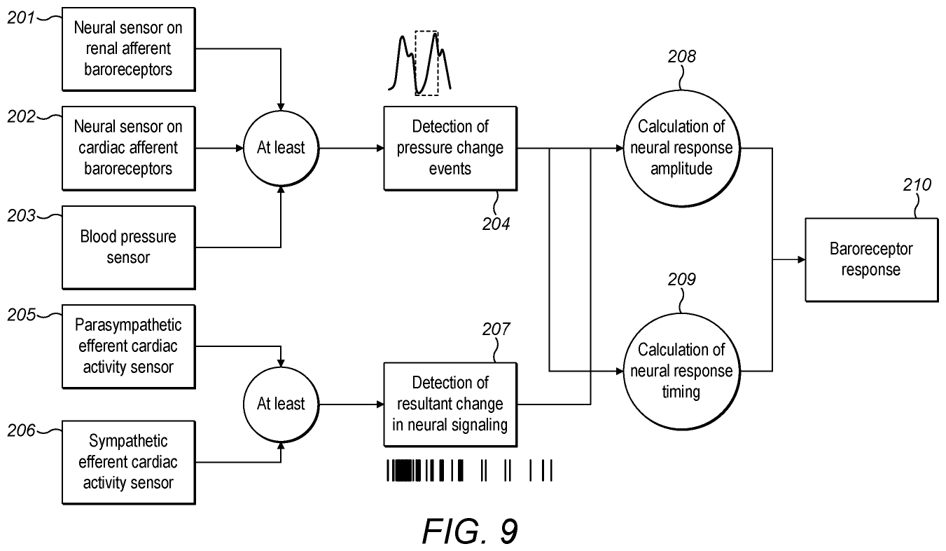

An example of a method of determining baroreceptor sensitivity which may be used in the cardiac control system of the present invention is shown schematically in FIG. 9.

In FIG. 9 a method 200 of determining baroreceptor sensitivity which may be used in the third embodiment is shown.

In the method 200, data regarding blood pressure change events is gathered by at least one of: at least one neural transducer 201 gathering neural data from at least one afferent nerve associated with a renal baroreceptor; at least one neural transducer 202 gathering neural data from at least one afferent nerve associated with a cardiac baroreceptor; and at least one blood pressure sensor 203 gathering blood pressure data. The gathered data regarding blood pressure change events is then processed in a detection step 204 to detect the timing and amplitude of sensed blood pressure change events.

In examples where the data regarding blood pressure change events is neural data gathered by a neural transducer the detection step 204 may comprise the use of an ML model, as discussed above.

Simultaneously, neural data relating to cardiac activity is gathered by at least one of: at least one parasympathetic efferent cardiac activity sensor 205 gathering neural data from at least one parasympathetic efferent cardiac nerve; and at least one sympathetic efferent cardiac activity sensor 206 gathering neural data from at least one sympathetic efferent cardiac nerve. The at least one parasympathetic efferent cardiac activity sensor 205 and the at least one sympathetic efferent cardiac activity sensor 206 may comprise suitably located neural transducers. The gathered data regarding cardiac activity is then processed in a detection step 207 to detect the changes in neural signaling from the brain stem resulting from the blood pressure change events and determine the changes in the timing and amplitude of heart rate which correspond to these changes in neural signaling.

The neural data gathered by the sensors 205 and/or 206 may be processed in the detection step 207 may comprise the use of an ML model, as discussed above.

Then, the amplitude of sensed blood pressure change events detected in the detection step 204 and the amplitude of changes in heart rate determined in the detection step 207 are compared to calculate a neural response amplitude in a neural response amplitude step 208, and the timing of sensed blood pressure change events detected in the detection step 204 and the timing of changes in heart rate determined in the detection step 207 are compared to calculate a neural response timing in a neural response timing step 209.

Then, the calculated neural response amplitude and the calculated a neural response timing are used to calculate the baroreceptor sensitivity in a baroreceptor sensitivity calculation step 210.

The calculated baroreceptor sensitivity may then be used by the cardiac control system in it's calculations. In other examples the calculated barometric sensitivity may be output to other systems.

The embodiments described above relate to a cardiac control system. The cardiac control system may be arranged to control two or more separate cardiac functions relating to separate diseases or conditions simultaneously. Further, the system is not limited to being only a cardiac control system, in some examples the output signals may be arranged to provide neural stimulation which will additionally bring the function of another organ of the subject closer to that of a healthy subject.

In the examples described above models are used, and these models may be machine learning (ML) models. In other examples alternative types of model may be used. In other examples the processing of neural data may be carried out without the use of models.

In the illustrated examples the controller comprises a communications module. In other examples the communications functions may be provided by a communications module or device separate from the controller.

In the examples described above the cardiac control system outputs control signals to neural stimulators 5. In alternative examples the cardiac control system may instead output the identified neural biomarkers identified by the ML neural processing. The output identified neural biomarkers may, for example, be sent to a device able to process the neural biomarkers and use them as the basis for neural stimulation signals to be applied to the subjects nervous system.

In some examples a targeted neural stimulus site to be stimulated by a neural stimulator may be treated with a viral vector or pharmaceutical agent arranged to enable hypersensitivity or hyposensitivity to neurostimulation.

In the illustrated examples all parts of the cardiac control system are implanted in the body of the subject. In some other examples neural transducers and stimulators may be implanted in a body of a subject, with the controller 3 of the cardiac control system 1 being carried out outside the body of the subject.

In some examples at least some parts of the cardiac control system which are implanted in the body of the subject have external surfaces of biocompatible materials.

In some examples the cardiac control system may be used to carry out autonomic control of cardiac function. In some examples the cardiac control system may be used to carry out PID control.

In some examples the neural data gathering and neural stimulation may be carried out entirely on the same implanted device and electrodes, on the same chip with different electrode contacts, different electrodes and different chips but with chips or electrodes housed in the same casing, or entirely separate. The sending of information to the external systems may be by way of a local base station connected to when at home or during charging or may be a direct connection over cellular or wifi data connections.

In some examples the neural transducers and neural stimulators may be arranged to operate using different modalities in order to eliminate cross-talk between the neural stimulators and the neural transducers. For example, the neural transducers can operate by electrical sensing while the neural stimulators operate by optogenetic stimulation.

In the illustrated examples the controller comprises a single data store. In alternative examples the single data store may be replaced by multiple data stores. In particular, in some examples there may be a dedicated data store for each of multiple different types of data.

In the illustrated examples the controller comprises a single processor. In other examples multiple processors may be used. In some examples a dedicated machine learning processor may be used to carry out machine learning model based processes.

The modules of the controller may be defined in software and/or in hardware.

In the illustrated examples cardiac control system has a single comptroller formed as a unitary device. This is not essential, in other examples the cardiac control system may comprise multiple controllers, and the one or multiple controllers may be formed as a distributed system.

In the examples described above the system is a closed loop cardiac control system. This is not essential. In some examples the system may be a cardiac control system without providing closed loop control.

In the described examples the system elements may be implemented as any form of a computing and/or electronic device.

The controller may comprise one or more processors which may be microprocessors, controllers or any other suitable type of processors for processing computer executable instructions to control the operation of the device in order to gather and record routing information. In some examples, for example where a system on a chip architecture is used, the processors may include one or more fixed function blocks (also referred to as accelerators) which implement a part of the method in hardware (rather than software or firmware). Platform software comprising an operating system or any other suitable platform software may be provided at the computing-based device to enable application software to be executed on the device.

The computer executable instructions may be provided using any computer-readable media that is accessible by computing based device. Computer-readable media may include, for example, computer storage media such as a memory and communications media. Computer storage media, such as a memory, includes volatile and non-volatile, removable and non-removable media implemented in any method or technology for storage of information such as computer readable instructions, data structures, program modules or other data. Computer storage media includes, but is not limited to, RAM, ROM, EPROM, EEPROM, flash memory or other memory technology, CD-ROM, digital versatile disks (DVD) or other optical storage, magnetic cassettes, magnetic tape, magnetic disk storage or other magnetic storage devices, or any other non-transmission medium that can be used to store information for access by a computing device. In contrast, communication media may embody computer readable instructions, data structures, program modules, or other data in a modulated data signal, such as a carrier wave, or other transport mechanism. As defined herein, computer storage media does not include communication media.

The term ‘computer’ is used herein to refer to any device with processing capability such that it can execute instructions. Those skilled in the art will realise that such processing capabilities are incorporated into many different devices and therefore the term ‘computer’ includes PCs, servers, mobile telephones, personal digital assistants and many other devices.

Those skilled in the art will realise that storage devices utilised to store program instructions can be distributed across a network. For example, a remote computer may store an example of the process described as software. A local or terminal computer may access the remote computer and download a part or all of the software to run the program. Alternatively, the local computer may download pieces of the software as needed, or execute some software instructions at the local terminal and some at the remote computer (or computer network). Those skilled in the art will also realise that by utilising conventional techniques known to those skilled in the art that all, or a portion of the software instructions may be carried out by a dedicated circuit, such as a DSP, programmable logic array, or the like.

It will be understood that the benefits and advantages described above may relate to one embodiment or may relate to several embodiments. The embodiments are not limited to those that solve any or all of the stated problems or those that have any or all of the stated benefits and advantages.

Any reference to ‘an’ item refers to one or more of those items. The term ‘comprising’ is used herein to mean including the method steps or elements identified, but that such steps or elements do not comprise an exclusive list and a method or apparatus may contain additional steps or elements.

The order of the steps of the methods described herein is exemplary, but the steps may be carried out in any suitable order, or simultaneously where appropriate. Additionally, steps may be added or substituted in, or individual steps may be deleted from any of the methods without departing from the scope of the subject matter described herein. Aspects of any of the examples described above may be combined with aspects of any of the other examples described to form further examples without losing the effect sought.

It will be understood that the above description of preferred embodiments is given by way of example only and that various modifications may be made by those skilled in the art. Although various embodiments have been described above with a certain degree of particularity, or with reference to one or more individual embodiments, those skilled in the art could make numerous alterations to the disclosed embodiments without departing from the spirit or scope of this invention.

1. A system for controlling a cardiac system of a subject, comprising:
a plurality of sensors arranged to detect physiological activity in a subject and produce physiological signals corresponding to the detected physiological activity;
at least one controller arranged to receive the physiological signals and to process the physiological signals using at least one model to determine at least one output signal; and
a plurality of neural stimulators arranged to receive the at least one output signal and to provide neural stimulation to the nervous system of the subject based on the at least one output signal.

2. The system of claim 1, wherein the plurality of sensors comprise a plurality of neural transducers arranged to detect neural activity in the nervous system of the subject and to produce physiological signals comprising neural data signals corresponding to the detected neural activity:
wherein the at least one controller is arranged to process the neural data signals using the at least one model to determine the at least one output signal to control cardiac function of the subject, whereby the provided neural stimulation modifies the cardiac function of the subject to either:
move one or more parameters of the cardiac function towards one or more predetermined values or ranges of values; or
to maintain one or more parameters of the cardiac function not to pass one or more predetermined limits; or
to produce a predetermined physiological response of the subject.

3. The system of claim 2, wherein the at least one controller is arranged to use at least one model to determine the at least one output signal to control cardiac function of the subject, whereby the provided neural stimulation modifies the cardiac function of the subject to be maintained within a defined operating range with predetermined limits.

4. The system of claim 3, wherein the at least one controller is arranged to set the predetermined limits of the defined operating range for the individual subject.

5. The system of claim 3 or claim 4, wherein the at least one controller is arranged to set the predetermined limits of the defined operating range by processing the neural data signals using at least one model.

6. The system of claim 2, wherein the provided neural stimulation modifies the cardiac function of the subject to produce a predetermined physiological response of the subject, and the predetermined physiological response is a measure associated with a return to healthy function of the cardiovascular system.

7. The system of claim 6, wherein the measure is one or more of: a reduction in mean blood pressure; reduction in at least one component of blood pressure; an increase in ejection fraction; an increase in pulse wave velocity.

8. The system of any preceding claim, wherein the controller is arranged to:
use at least one model to determine a desired operating point of the cardiac system of the subject;
make a control decision to change the current operating point of the cardiac system of the subject towards the determined desired operating point; and
use at least one model to determine at least one output signal to move one or more parameters of the cardiac function towards the desired operating point of the cardiac system of the subject according to the control decision.

9. The system of claim 8, wherein the model is constrained to keep the desired operating point of the cardiac system to be one or more predetermined values, or to be maintained not to pass one or more predetermined limits.

10. The system of any preceding claim, wherein the plurality of neural stimulators are arranged to provide neural stimulation to the nervous system of the subject at, at least one of: brain stem; upper spinal cord; cardiac sympathetic branches; renal sympathetic branches; upper Vagus nerve; cardiac branch of Vagus nerve; renal branch of Vagus nerve.

11. The system of claim 10, wherein the plurality of neural stimulators are arranged to modify neural activity relating to at least one of sympathetic afferent neural signals or sympathetic efferent neural signals going to at least one of a cardiac system or a renal system of the subject.

12. The system of claim 11, wherein the plurality of neural stimulators are arranged to modify nerve activity to inhibit nerve activity or to stimulate nerve activity.

13. The system of any preceding claim, wherein the plurality of sensors comprise a plurality of neural transducers arranged to detect neural activity in the nervous system of the subject at, at least one of: brain stem; upper spinal cord; cardiac sympathetic branches; renal sympathetic branches; upper Vagus nerve; cardiac branch of Vagus nerve; renal branch of Vagus nerve; barroreceptors; muscle afferent nerves.

14. The system of claim 13, wherein the plurality of neural transducers are arranged to detect neural activity relating to at least one of sympathetic afferent neural signals or sympathetic efferent neural signals going to at least one of a cardiac system or a renal system of the subject.

15. The system of any preceding claim, wherein:
the plurality of sensors comprise a plurality of neural transducers arranged to detect neural activity in the nervous system of the subject at the Vagus nerve and/or the spinal cord of the subject; and
the plurality of neural stimulators are arranged to provide neural stimulation to the Vagus nerve and/or the spinal cord of the subject.

16. The system of any preceding claim, wherein the plurality of sensors comprise a plurality of neural transducers arranged to detect neural activity in the nervous system of the subject at the Vagus nerve and to produce physiological signals comprising neural data signals corresponding to this detected neural activity; and
the at least one controller is arranged to process these neural data signals to determine information regarding cardiac parasympathetic activity of the subject.

17. The system of claim 16, wherein the plurality of neural transducers are arranged to detect neural activity in a cardiac branch of the Vagus nerve of the subject.

18. The system of any preceding claim, wherein the plurality of sensors comprise a plurality of neural transducers arranged to detect neural activity in the nervous system of the subject at the spinal cord and to produce physiological signals comprising neural data signals corresponding to this detected neural activity; and
the at least one controller is arranged to process these neural data signals to determine information regarding cardiac sympathetic activity of the subject.

19. The system of claim 18, wherein the plurality of neural transducers are arranged to detect neural activity cranial to the T4 vertabrae of the subject.

20. The system of any one of claims 15 to 19, wherein the plurality of sensors further comprise a plurality of neural transducers arranged to detect neural activity in the nervous system of the subject at one or more of: carotid baroreceptors; aortic baroreceptors; renal afferent nerves; muscular afferent nerves.

21. The system of any preceding claim, wherein the plurality of neural stimulators are arranged to provide neural stimulation to sympathetic cardiac neural pathways in the spinal cord of the subject.

22. The system of any preceding claim, wherein the plurality of neural stimulators are arranged to provide neural stimulation to sympathetic renal neural pathways in the spinal cord of the subject.

23. The system of any preceding claim, wherein the plurality of neural stimulators are arranged to provide neural stimulation to the spinal cord cranial to the T4 vertabrae of the subject.

24. The system of any preceding claim, wherein the plurality of neural stimulators are arranged to provide neural stimulation to parasympathetic renal neural pathways in the Vagus nerve of the subject.

25. The system of any preceding claim, wherein the plurality of neural stimulators are arranged to provide neural stimulation to parasympathetic renal neural pathways in the Vagus nerve of the subject.

26. The system of any preceding claim, wherein one, some, or all of the plurality of neural stimulators are arranged to provide neural stimulation which blocks natural neural activity, either wholly or in part.

27. The system of claim 26, wherein one, some, or all of the plurality of neural stimulators are arranged to provide neural stimulation at a frequency in the range 5 kHz to 30 kHz to block natural neural activity.

28. The system of claim 26 or claim 27, wherein the controller is arranged to determine at least one output signal which causes one, some, or all of the plurality of neural stimulators to block natural neural activity on a nerve in response to detection of natural efferent neural activity on that nerve that would move one or more parameters of the cardiac function of the subject past one or more predetermined values or away from a desired operating point.

29. The system of any preceding claim, wherein one, some, or all of the plurality of neural stimulators are arranged to provide neural stimulation which at least partially modifies natural neural activity.

30. The system of any preceding claim, wherein one, some, or all of the plurality of neural stimulators are arranged to provide neural stimulation which at least partially amplifies natural neural activity.

31. The system of any preceding claim, wherein one, some, or all of the plurality of neural stimulators are arranged to provide neural stimulation producing an applied neural signal which is additional to natural neural signals.

32. The system of any preceding claim, wherein the plurality of neural stimulators are arranged to provide at least one of: electrical stimulation; chemical activation; mechanical stimulation; ultrasonic stimulation; thermal stimulation; and/or optogenic stimulation.

33. The system of any preceding claim, wherein the controller is further arranged to receive a heart signal identifying electrical activity of the subjects heart and to process the heart signal together with the neural data signals using the at least one model to determine the at least one output signal.

34. The system of claim 33, wherein the heart signal identifies electrical activity of the subjects heart comprising at least one of: Heart Rate, Heart Rate Variability, P wave shape, T wave duration, T wave amplitude, J point, ST elevation, U wave, R-R interval, signal period, frequency profile, amplitude, or other relevant features derived from the signal identifying electrical activity of the heart.

35. The system of claim 34, wherein the at least one controller is arranged to process the heart signal using at least one model to generate estimates of features of the blood pressure including at least one of: Systolic pressure, Diastolic pressure, peak pressure, mean blood pressure, Ejection Time, Pulse Wave Velocity, or other relevant features derived from the signal identifying the blood pressure in the cardiac system.

36. The system of any one of claims 33 to 35, wherein the system further comprises one or more electrical sensors arranged to sense electrical activity of the subjects heart and to generate the heart signal.

37. The system of any preceding claim, wherein the controller is further arranged to receive a blood pressure signal identifying a blood pressure of the subject and to process the blood pressure signal together with the neural data signals using the at least one model to determine the at least one output signal.

38. The system of claim 37, wherein the blood pressure signal identifies blood pressure of the subject comprising at least one of: Systolic pressure, Diastolic pressure, peak pressure, Orthostatic drop, mean blood pressure, Ejection Time, Pulse Wave Velocity, or other relevant features derived from the signal identifying the blood pressure in the cardiac system.

39. The system of claim 37 or claim 38, wherein the system further comprises one or more blood pressure sensors arranged to sense blood pressure of the subject and to generate the blood pressure signal.

40. The system of any of claims 33 to 37, wherein the at least one controller is further arranged to receive both of the heart signal identifying electrical activity of the subjects heart and the blood pressure signal identifying a blood pressure of the subject and to process the heart electrical signal together with the blood pressure signal and the neural data signals using at least one model to determine the at least one output signal.

41. The system of claim 40, wherein the at least one controller is further arranged to receive both of the heart signal identifying electrical activity of the subjects heart and the blood pressure signal identifying a blood pressure of the subject and to process the heart electrical signal together with the blood pressure signal to identify cardiac activity of the subject comprising at least one of: Q-A interval, Baroreceptor Sensitivity, Volumetric Cardiac Output or other relevant features derived from joint cross-analysis of the heart electrical signal and blood pressure signal.

42. The system of any preceding claim, wherein the controller is further arranged to receive further neural data signals corresponding to neural activity associated with one or more of: carotid baroreceptors; aortic baroreceptors; renal afferent nerves; and muscular afferent nerves.

43. The system of claim 42, wherein the plurality of neural sensors further comprises neural sensors arranged to detect neural activity associated with one or more of: carotid baroreceptors; aortic baroreceptors; renal afferent nerves; and muscular afferent nerves, and to produce the further neural data signals.

44. The system of any preceding claim, wherein the controller is arranged to provide the at least one output signal to the plurality of neural stimulators in response to identification of a predetermined event.

45. The system of claim 44, wherein the predetermined event is a neural event.

46. The system of claim 45, wherein the predetermined event is at least one of: baroreceptor firing indicative of blood pressure changes; renal afferent firing indicative of low blood perfusion; sympathetic firing indicative of cardiac upregulation.

47. The system of claim 44, wherein the predetermined event is a non-neural event.

48. The system of claim 47, wherein the predetermined event is at least one of: heart rate too high; heart rate too low; blood glucose too high; blood glucose too low.

49. The system of claim 37 or claim 38, wherein the controller is arranged to provide the at least one output signal to the plurality of neural stimulators in response to identification of a blood pressure of the subject exceeding a predetermined threshold value.

50. The system of any preceding claim, wherein the system is a closed loop cardiac control system.

51. The system of any preceding claim, wherein the system is implanted within a body of the subject.

52. The system of claim 51, wherein the system comprises external surfaces of biocompatible materials.

53. The system of any preceding claim, wherein the at least one model comprises an updating model predictive controller.

54. The system of any preceding claim, wherein the at least one model comprises a machine learning model.

55. The system of any preceding claim, wherein the controller is arranged to receive updates to the one or more models from an external system.

56. The system of any preceding claim, wherein the neural stimulation applied to the nervous system of the subject for controlling the cardiac system of the subject additionally brings the function of another organ of the subject closer to that of a healthy subject.

57. The system of any preceding claim, wherein the system is updated to change which cardiac function parameters it is controlling.

58. The system of any preceding claim, wherein the system controls two or more separate cardiac functions relating to separate diseases simultaneously.

59. The system of any preceding claim, wherein the at least one controller is arranged to process neural data signals with:
at least one first model arranged to determine at least one output signal to bring cardiac function of the subject closer to that of a healthy subject; and
a second model arranged to determine at least one output signal to bring the function of another organ closer to that of a healthy subject.

60. A system comprising a closed loop cardiac function control system according to any preceding claim and an external system.

61. The system according to claim 60, wherein the external system comprises at least one machine learning means arranged to generate updates to the at least one model by machine learning and to send the generated updates to the at least one model to the closed loop cardiac function control system.

62. The system according to claim 60 or claim 61, wherein the external system comprises at least one reporting system arranged to receive data regarding the operation of the cardiac control system and data regarding the subject from the controller, and to calculate subject outcome measures for the cardiac control system.

63. The system according to any one of claims 60 to 62, wherein the external system comprises at least one treatment information system arranged to inform the cardiac control system regarding treatments being provided to the subject.

64. The system according to any one of claims 60 to 63, wherein the external system comprises at least one security system arranged to control access to personal information regarding the subject which is held by the external system.

65. A method for carrying out closed loop cardiac function control comprising;
implanting a system according to any one of claims 1 to 59 into a body of a subject; and
operating the system.

66. A system configured to modulate efferent neural activity of at least one cardiac sympathetic nerve of a subject, the system comprising:
at least one controller arranged to determine at least one output signal; and
a plurality of neural stimulators arranged to apply the at least one output signal to the at least one cardiac sympathetic nerve of the subject;
wherein the at least one output signal modulates the efferent neural activity of the at least one cardiac sympathetic nerve to produce a physiological response in the subject.

67. The system of claim 66, wherein the at least one cardiac sympathetic nerve is in the spinal cord.

68. The system of claim 66, wherein the at least one cardiac sympathetic nerve is in the cardiac sympathetic branches going from the spinal cord to the heart.

69. The system of any one of claims 66 to 68, wherein the physiological response is a measure associated with a return to healthy function of the cardiovascular system of the subject.

70. The system of claim 69, wherein the physiological response is one or more of: a reduction in blood pressure; an increase in ejection fraction.

71. The system of any one of claims 66 to 70, wherein the at least one output signal is arranged to provide neural stimulation which at least partially modifies natural neural activity.

72. The system of any one of claims 66 to 71, wherein the at least one output signal is arranged to provide neural stimulation which at least partially amplifies natural neural activity.

73. The system of any one of claims 66 to 72, wherein the at least one output signal is arranged to provide neural stimulation producing an applied neural signal which is additional to natural neural signals.

74. The system of any one of claims 66 to 70, wherein at least one output signal is arranged to provide neural stimulation which blocks natural neural activity, either wholly or in part.

75. The system of claim 74, wherein the at least one output signal is at a frequency in the range 5 kHz to 30 kHz to block natural neural activity.

76. The system of any one of claims 66 to 75, wherein the at least one output signal comprises at least one of: electrical stimulation; chemical activation; mechanical stimulation; ultrasonic stimulation; thermal stimulation; and/or optogenic stimulation.

77. The system of any one of claims 66 to 76, wherein the at least one controller determines the at least one output signal by processing detected neural activity of the subject using a model.

78. The system of claim 77, wherein the model is a machine learning model.

79. The system according to claim 76 or claim 77, wherein the system is a closed loop control system.

80. A system configured to modulate efferent neural activity of at least one renal sympathetic nerve of a subject, the system comprising:
at least one controller arranged to determine at least one output signal; and
a plurality of neural stimulators arranged to apply the output signal to the at least one renal sympathetic nerve of the subject;
wherein the output signal modulates the efferent neural activity of the at least one renal sympathetic nerve to produce a physiological response in the subject.

81. The system of claim 80, wherein the at least one renal sympathetic nerve is in the spinal cord.

82. The system of claim 80, wherein the at least one renal sympathetic nerve is in the renal sympathetic branches going from the spinal cord to the renal system.

83. The system of any one of claims 80 to 82, wherein the physiological response is a measure associated with a return to health function of the cardiovascular system of the subject.

84. The system of claim 83, wherein the physiological response is one or more of: a reduction in blood pressure; an increase in ejection fraction.

85. The system of any one of claims 80 to 84, wherein the at least one output signal is arranged to provide neural stimulation which at least partially modifies natural neural activity.

86. The system of any one of claims 80 to 85, wherein the at least one output signal is arranged to provide neural stimulation which at least partially amplifies natural neural activity.

87. The system of any one of claims 80 to 86, wherein the at least one output signal is arranged to provide neural stimulation producing an applied neural signal which is additional to natural neural signals.

88. The system of any one of claims 80 to 84, wherein at least one output signal is arranged to provide neural stimulation which blocks natural neural activity, either wholly or in part.

89. The system of claim 88, wherein the at least one output signal is at a frequency in the range 5 kHz to 30 kHz to block natural neural activity.

90. The system of any one of claims 80 to 89, wherein the at least one output signal comprises at least one of: electrical stimulation; chemical activation; mechanical stimulation; ultrasonic stimulation; thermal stimulation; and/or optogenic stimulation.

91. The system of any one of claims 80 to 90, wherein the at least one controller determines the at least one output signal by processing detected neural activity of the subject using a model.

92. The system of claim 91, wherein the model is a machine learning model.

93. The system according to claim 91 or claim 92, wherein the system is a closed loop control system.

94. A system configured to modulate efferent neural activity of at least one cardiac parasympathetic nerve of a subject, the system comprising:
at least one controller arranged to determine at least one output signal; and
a plurality of neural stimulators arranged to apply the output signal to the at least one cardiac parasympathetic nerve of the subject;
wherein the output signal modulates the efferent neural activity of the at least one cardiac parasympathetic nerve to produce a physiological response in the subject.

95. The system of claim 94, wherein the at least one cardiac parasympathetic nerve fibre is in the Vagus nerve.

96. The system of claim 94, wherein the at least one cardiac parasympathetic nerve fibre is in a cardiac branch of the Vagus nerve.

97. The system of any one of claims 94 to 96, wherein the physiological response is a measure associated with a return to health function of the cardiovascular system of the subject.

98. The system of claim 97, wherein the physiological response is one or more of: a reduction in blood pressure; an increase in ejection fraction.

99. The system of any one of claims 94 to 98, wherein the at least one output signal is arranged to provide neural stimulation which at least partially modifies natural neural activity.

100. The system of any one of claims 94 to 99, wherein the at least one output signal is arranged to provide neural stimulation which at least partially amplifies natural neural activity.

101. The system of any one of claims 94 to 100, wherein the at least one output signal is arranged to provide neural stimulation producing an applied neural signal which is additional to natural neural signals.

102. The system of any one of claims 94 to 98, wherein at least one output signal is arranged to provide neural stimulation which blocks natural neural activity, either wholly or in part.

103. The system of claim 102, wherein the at least one output signal is at a frequency in the range 5 kHz to 30 kHz to block natural neural activity.

104. The system of any one of claims 94 to 103, wherein the at least one output signal comprises at least one of: electrical stimulation; chemical activation; mechanical stimulation; ultrasonic stimulation; thermal stimulation; and/or optogenic stimulation.

105. The system of any one of claims 94 to 104, wherein the at least one controller determines the at least one output signal by processing detected neural activity of the subject using a model.

106. The system of claim 105, wherein the model is a machine learning model.

107. The system according to claim 105 or claim 106, wherein the system is a closed loop control system.

108. A system configured to modulate afferent neural activity of at least one nerve associated with baroreceptors of a subject, the system comprising:
at least one controller arranged to determine at least one output signal; and
a plurality of neural stimulators arranged to apply the output signal to the at least one nerve associated with at least one baroreceptor of the subject;
wherein the output signal modulates the afferent neural activity of the at least one nerve associated with the at least one baroreceptor to produce a physiological response in the subject.

109. The system of claim 108, wherein the at least one nerve associated with the at least one baroreceptor is in the Vagus nerve.

110. The system of claim 108, wherein the at least one nerve associated with the at least one baroreceptor is the carotid sinus nerve.

111. The system of claim 108, wherein the at least one nerve associated with the at least one baroreceptor is the glossopharyngeal nerve.

112. The system of claim 108, wherein the at least one nerve associated with the at least one baroreceptor are renal parasympathetic nerve fibres in the vagus or pelvic nerves.

113. The system of any one of claims 108 to 112, wherein the physiological response is a measure associated with a return to health function of the cardiovascular system of the subject.

114. The system of claim 113, wherein the physiological response is one or more of: a reduction in blood pressure; an increase in ejection fraction.

115. The system of any one of claims 108 to 114, wherein the at least one output signal is arranged to provide neural stimulation which at least partially modifies natural neural activity.

116. The system of any one of claims 108 to 115, wherein the at least one output signal is arranged to provide neural stimulation which at least partially amplifies natural neural activity.

117. The system of any one of claims 108 to 116, wherein the at least one output signal is arranged to provide neural stimulation producing an applied neural signal which is additional to natural neural signals.

118. The system of any one of claims 108 to 114, wherein at least one output signal is arranged to provide neural stimulation which blocks natural neural activity, either wholly or in part.

119. The system of claim 118, wherein the at least one output signal is at a frequency in the range 5 kHz to 30 kHz to block natural neural activity.

120. The system of any one of claims 108 to 119, wherein the at least one output signal comprises at least one of: electrical stimulation; chemical activation; mechanical stimulation; ultrasonic stimulation; thermal stimulation; and/or optogenic stimulation.

121. The system of any one of claims 108 to 120, wherein the at least one controller determines the at least one output signal by processing detected neural activity of the subject using a model.

122. The system of claim 121, wherein the model is a machine learning model.

123. The system according to claim 121 or claim 122, wherein the system is a closed loop control system.

124. A system configured to determine cardiac activity of a subject, the system comprising:
at least one neural transducer arranged to receive efferent neural activity of at least one cardiac sympathetic nerve of the subject, and to produce neural data signals derived from the received efferent neural activity;
at least one processor arranged to process the neural data signals to provide processed neural data signals, and to use the processed neural data signals to determine cardiac function of the subject.

125. The system of claim 124, wherein the at least one processor comprises at least one model arranged to process the neural data signals to provide processed neural data signals.

126. The system of claim 125, wherein the at least one model is at least one machine learning model.

127. The system of any one of claims 124 to 126, wherein the at least one processor is arranged to process the neural data signals to identify one or more neural biomarkers from the neural data signals.

128. The system of any one of claims 124 to 127, wherein the at least one cardiac sympathetic nerve is in the spinal cord.

129. The system of any one of claims 124 to 128, wherein the at least one cardiac sympathetic nerve is in the cardiac sympathetic branches going to the heart.

130. The system of any one of claims 124 to 129, wherein the at least one processor is arranged to use the processed neural data signals to inform one or more cardiovascular models of the sympathetic drive of the heart.

131. The system of any one of claims 124 to 130, wherein the at least one processor is arranged to use the processed neural data signals to determine current cardiac activity of the subject.

132. The system of any one of claims 124 to 131, the system further comprising:
at least one controller arranged to use the determined cardiac activity of the subject to determine at least one output signal; and
a plurality of neural stimulators arranged to apply the at least one output signal to a nervous system of the subject to produce a physiological response in the subject.

133. The system of claim 132, wherein the physiological response is a measure associated with a return to health function of the cardiovascular system of the subject.

134. The system of claim 133, wherein the physiological response is one or more of: a reduction in mean blood pressure; reduction in at least one component of blood pressure; an increase in ejection fraction, an increase in pulse wave velocity.

135. The system of any one of claims 132 to 134, wherein the at least one output signal is arranged to provide neural stimulation which at least partially modifies natural neural activity.

136. The system of any one of claims 132 to 135, wherein the at least one output signal is arranged to provide neural stimulation which at least partially amplifies natural neural activity.

137. The system of any one of claims 132 to 136, wherein the at least one output signal is arranged to provide neural stimulation producing an applied neural signal which is additional to natural neural signals.

138. The system of any one of claim 132 or 133, wherein at least one output signal is arranged to provide neural stimulation which blocks natural neural activity, either wholly or in part.

139. The system of claim 138, wherein the at least one output signal is at a frequency in the range 5 kHz to 30 kHz to block natural neural activity.

140. The system of any one of claims 132 to 139, wherein the at least one output signal comprises at least one of: electrical stimulation; chemical activation; mechanical stimulation; ultrasonic stimulation; thermal stimulation; and/or optogenic stimulation.

141. The system of any one of claims 132 to 140, wherein the at least one controller determines the at least one output signal by processing detected neural activity of the subject using a model.

142. The system of claim 141, wherein the model is a machine learning model.

143. A system configured to determine cardiovascular activity of a subject, the system comprising:
at least one neural transducer arranged to receive efferent neural activity of at least one renal sympathetic nerve of the subject, and to produce neural data signals derived from the received efferent neural activity;
at least one processor arranged to process the neural data signals to provide processed neural data signals, and to use the processed neural data signals to determine cardiovascular activity of the subject.

144. The system of claim 143, wherein the at least one processor comprises at least one model arranged to process the neural data signals to provide processed neural data signals.

145. The system of claim 144, wherein the at least one model is at least one machine learning model.

146. The system of any one of claims 143 to 145, wherein the at least one processor is arranged to process the neural data signals to identify one or more neural biomarkers from the neural data signals.

147. The system of any one of claims 143 to 146, wherein the at least one renal sympathetic nerve is in the spinal cord.

148. The system of any one of claims 143 to 146, wherein the at least one renal sympathetic nerve is in the renal sympathetic branches going to the kidneys.

149. The system of any one of claims 143 to 148, wherein the at least one processor is arranged to use the processed neural data signals to inform one or more cardiovascular models of the sympathetic drive of the renal system.

150. The system of any one of claims 143 to 149, wherein the at least one processor is arranged to use the processed neural data signals to determine current cardiovascular activity of the subject.

151. The system of any one of claims 143 to 150, the system further comprising:
at least one controller arranged to use the determined cardiovascular activity of the subject to determine at least one output signal; and
a plurality of neural stimulators arranged to apply the at least one output signal to a nervous system of the subject to produce a physiological response in the subject.

152. The system of claim 151, wherein the physiological response is a measure associated with a return to health function of the cardiovascular system of the subject.

153. The system of claim 152, wherein the physiological response is one or more of: a reduction in mean blood pressure; reduction in at least one component of blood pressure; an increase in ejection fraction, an increase in pulse wave velocity.

154. The system of any one of claims 143 to 153, wherein the at least one output signal is arranged to provide neural stimulation which at least partially modifies natural neural activity.

155. The system of any one of claims 143 to 154, wherein the at least one output signal is arranged to provide neural stimulation which at least partially amplifies natural neural activity.

156. The system of any one of claims 143 to 155, wherein the at least one output signal is arranged to provide neural stimulation producing an applied neural signal which is additional to natural neural signals.

157. The system of any one of claims 143 to 156, wherein at least one output signal is arranged to provide neural stimulation which blocks natural neural activity, either wholly or in part.

158. The system of claim 157, wherein the at least one output signal is at a frequency in the range 5 kHz to 30 kHz to block natural neural activity.

159. The system of any one of claims 151 to 158, wherein the at least one output signal comprises at least one of: electrical stimulation; chemical activation; mechanical stimulation; ultrasonic stimulation; thermal stimulation; and/or optogenic stimulation.

160. The system of any one of claims 151 to 159, wherein the at least one controller determines the at least one output signal by processing detected neural activity of the subject using a model.

161. The system of claim 160, wherein the model is a machine learning model.

162. A system configured to determine cardiovascular activity of a subject, the system comprising:
at least one neural transducer arranged to receive afferent neural activity of at least one renal nerve of the subject, and to produce neural data signals derived from the received efferent neural activity;
at least one processor arranged to process the neural data signals to provide processed neural data signals, and to use the processed neural data signals to determine cardiovascular activity of the subject.

163. The system of claim 162, wherein the at least one processor comprises at least one model arranged to process the neural data signals to provide processed neural data signals.

164. The system of claim 163, wherein the at least one model is at least one machine learning model.

165. The system of any one of claims 162 to 164, wherein the at least one processor is arranged to process the neural data signals to identify one or more neural biomarkers from the neural data signals.

166. The system of any one of claims 162 to 165, wherein the at least one renal nerve is in the spinal cord.

167. The system of any one of claims 162 to 165, wherein the at least one renal nerve is in the Vagus nerve.

168. The system of any one of claims 162 to 165, wherein the at least one renal nerve is in the pelvic nerves.

169. The system of any one of claims 162 to 165, wherein the at least one renal nerve is in the renal sympathetic branches going to the kidneys.

170. The system of any one of claims 162 to 169, wherein the at least one processor is arranged to use the processed neural data signals to inform one or more cardiovascular models of at least one of: peripheral pressure, peripheral perfusion, hypertension disease progression, renal activity.

171. The system of any one of claims 162 to 170, wherein the at least one processor is arranged to use the processed neural data signals to determine current cardiovascular activity of the subject.

172. The system of any one of claims 162 to 171, the system further comprising:
at least one controller arranged to use the determined cardiovascular activity of the subject to determine at least one output signal; and
a plurality of neural stimulators arranged to apply the at least one output signal to a nervous system of the subject to produce a physiological response in the subject.

173. The system of claim 172, wherein the physiological response is a measure associated with a return to health function of the cardiovascular system of the subject.

174. The system of claim 173, wherein the physiological response is one or more of: a reduction in mean blood pressure; reduction in at least one component of blood pressure; an increase in ejection fraction, an increase in pulse wave velocity.

175. The system of any one of claims 172 to 174, wherein the at least one output signal is arranged to provide neural stimulation which at least partially modifies natural neural activity.

176. The system of any one of claims 174 to 175, wherein the at least one output signal is arranged to provide neural stimulation which at least partially amplifies natural neural activity.

177. The system of any one of claims 172 to 176, wherein the at least one output signal is arranged to provide neural stimulation producing an applied neural signal which is additional to natural neural signals.

178. The system of any one of claim 172 0r 173, wherein at least one output signal is arranged to provide neural stimulation which blocks natural neural activity, either wholly or in part.

179. The system of claim 178, wherein the at least one output signal is at a frequency in the range 5 kHz to 30 kHz to block natural neural activity.

180. The system of any one of claims 172 to 179, wherein the at least one output signal comprises at least one of: electrical stimulation; chemical activation; mechanical stimulation; ultrasonic stimulation; thermal stimulation; and/or optogenic stimulation.

181. The system of any one of claims 172 to 180, wherein the at least one controller determines the at least one output signal by processing detected neural activity of the subject using a model.

182. The system of claim 181, wherein the model is a machine learning model.

183. A system configured to receive signals associated with cardiac function of a subject, the system comprising:
at least one sensor arranged to produce at least one signal associated with blood pressure rise and fall of the subject; and
at least one sensor arranged to produce at least one signal associated with efferent neural activity to the heart of the subject;
wherein the system is arranged to register the timing and magnitude of changes in blood pressure;
wherein the system is arranged to register the timing and magnitude of natural efferent neural signals to the heart;
wherein the system is arranged to determine a relationship between timing of the natural efferent neural signals to the heart and timing of any corresponding blood pressure change; and
wherein the system is arranged to determine a relationship between a magnitude of the natural efferent neural signals and a magnitude of any corresponding blood pressure change.

184. The system of claim 183, wherein the received signal associated with blood pressure rise and fall is derived from afferent neural activity coming from baroreceptors of the subject.

185. The system of claim 183 or 184, where the received signals are sampled at a frequency greater than 10 Hz to capture beat to beat blood pressure rise and fall.

186. The system of any one of claims 183 to 185, wherein the at least one sensor arranged to produce at least one signal associated with blood pressure rise and fall of the subject is at least one neural transducer.

187. The system of any one of claims 183 to 186, wherein the at least one sensor arranged to produce at least one signal associated with efferent neural activity to the heart of the subject is at least one neural transducer.

188. The system of any one of claims 183 to 187, the system further comprising:
at least one controller arranged to use the received at least one signal associated with the blood pressure of the subject to determine at least one output signal; and
a plurality of neural stimulators arranged to apply the at least one output signal to a nervous system of the subject to produce a physiological response in the subject.

189. The system of claim 188, wherein the physiological response is a measure associated with a return to health function of the cardiovascular system of the subject.

190. The system of claim 189, wherein the physiological response is one or more of: a reduction in mean blood pressure; reduction in at least one component of blood pressure; an increase in ejection fraction, and increase in pulse wave velocity.

191. The system of any one of claims 188 to 190, wherein the timing of the at least one signal associated with the blood pressure rise and fall of the subject is used as the input signal for the controller.

192. The system of any one of claims 188 to 191, wherein the magnitude of the at least one signal associated with the blood pressure rise and fall of the subject is used as the input signal for the controller.

193. The system of any one of claims 188 to 192, wherein the at least one output signal is arranged to provide neural stimulation which at least partially modifies natural neural activity.

194. The system of any one of claims 188 to 193, wherein the at least one output signal is arranged to provide neural stimulation which at least partially amplifies natural neural activity.

195. The system of any one of claims 188 to 194, wherein the at least one output signal is arranged to provide neural stimulation producing an applied neural signal which is additional to natural neural signals.

196. The system of any one of claims 188 to 195, wherein at least one output signal is arranged to provide neural stimulation which blocks natural neural activity, either wholly or in part.

197. The system of claim 196, wherein the at least one output signal is at a frequency in the range 5 kHz to 30 kHz to block natural neural activity.

198. The system of any one of claims 188 to 197, wherein the at least one output signal comprises at least one of: electrical stimulation; chemical activation; mechanical stimulation; ultrasonic stimulation; thermal stimulation; and/or optogenic stimulation.

199. The system of any one of claims 188 to 198, wherein the at least one controller determines the at least one output signal by processing detected neural activity of the subject using a model.

200. The system of claim 199, wherein the model is a machine learning model.

201. The system of any one of claims 183 to 200, wherein the timing of the efferent neural activity to the heart relative to the timing of any blood pressure change is the delay of the baroreceptor response, known as the baroreceptor sensitivity

202. The system of any one of claims 183 to 201, wherein the magnitude of the efferent neural activity to the heart relative to the magnitude of any blood pressure change is the gain of the baroreceptor response.

203. A method of determining baroreceptor sensitivity of a subject, the method comprising:
receiving at least one signal associated with blood pressure rise and fall of the subject; and
receiving at least one signal associated with efferent neural activity to the heart of the subject;
registering the timing and magnitude of changes in blood pressure;
registering the timing and magnitude of natural efferent neural signals to the heart;
determining a timing relationship between timing of the natural efferent neural signals to the heart and timing of any corresponding blood pressure change; and
determining a magnitude relationship between a magnitude of the natural efferent neural signals and a magnitude of any corresponding blood pressure change; and
determining a baroreceptor sensitivity using the determined timing relationship and magnitude relationship.

204. The method of claim 203, wherein the received signal associated with blood pressure rise and fall is derived from afferent neural activity coming from baroreceptors of the subject.

205. The method of claim 203 or 204, where the received signals are sampled at a frequency greater than 10 Hz to capture beat to beat blood pressure rise and fall.

206. The method of any one of claims 203 to 205, wherein the at least one signal associated with blood pressure rise and fall of the subject is at least one neural data signal.

207. The method of any one of claims 203 to 206, wherein the at least one signal associated with efferent neural activity to the heart of the subject is at least one neural data signal.
